SEQUENCE LISTING (231 sequences)

SEQ ID NO: 1 - DNA, 33 bp, Homo Sapiens
  misc_feature: n equates to a insertion of “c to ca” or “g to ga”.
cgctggccatcccgccrgctggggggtgcacga

SEQ ID NO: 2 - DNA, 33 bp, Homo Sapiens
cagcaaaagcacaacakctggggaggagctagt

SEQ ID NO: 3 - DNA, 33 bp, Homo Sapiens
gaggaaaaccaagacasagcaaaaatttttaaa

SEQ ID NO: 4 - DNA, 33 bp, Homo Sapiens
cgaacagaggaggaacrgaagcataagctagaa

SEQ ID NO: 5 - DNA, 33 bp, Homo Sapiens
cctgctgtggattcctrtgcttctgggtgtcca

SEQ ID NO: 6 - DNA, 33 bp, Homo Sapiens
gagcgggacaccagccrgcggctgctggcggaa

SEQ ID NO: 7 - DNA, 33 bp, Homo Sapiens
catgcgcaagctggtgygctcagtgactgtggt

SEQ ID NO: 8 - DNA, 33 bp, Homo Sapiens
ggtcgaacttctctgcmtccaagttatagatgc

SEQ ID NO: 9 - DNA, 33 bp, Homo Sapiens
ttcattcaggtggtcartggccagcaccttcct

SEQ ID NO: 10 - DNA, 33 bp, Homo Sapiens
gccttcttcttccgggmctcatcctcagccttc

SEQ ID NO: 11 - DNA, 33 bp, Homo Sapiens
ggaaaggctgtactacmgtcttcgtcctctctc

SEQ ID NO: 12 - DNA, 33 bp, Homo Sapiens
ttcctaaaatgttggawgctgaaggtgagatga

SEQ ID NO: 13 - DNA, 33 bp, Homo Sapiens
gaggagtggttgctcartgagattcggagactg

SEQ ID NO: 14 - DNA, 33 bp, Homo Sapiens
tgaactggactatcacracgctgtgaatgtcaa

SEQ ID NO: 15 - DNA, 33 bp, Homo Sapiens
gattcgaggtgctttgrctgaagctcggaaaat

SEQ ID NO: 16 - DNA, 33 bp, Homo Sapiens
ctcgccaagtgtgaccragtggaccagctgaca

SEQ ID NO: 17 - DNA, 33 bp, Homo Sapiens
gcagtctcgctccttcygcatcctggcccagat

SEQ ID NO: 18 - DNA, 33 bp, Homo Sapiens
caccccgctgctgcccrcttctgcccagccacc

SEQ ID NO: 19 - DNA, 33 bp, Homo Sapiens
ccctgcacccaagcccsgggttgtcaccactgc

SEQ ID NO: 20 - DNA, 33 bp, Homo Sapiens
gcatctacctacagccsgtccccaggggccaat

SEQ ID NO: 21 - DNA, 33 bp, Homo Sapiens
ctcggtggcctacagcrggggccctgcggagcc

SEQ ID NO: 22 - DNA, 33 bp, Homo Sapiens
acttgccagggggaccrtccagagggctgagcg

SEQ ID NO: 23 - DNA, 33 bp, Homo Sapiens
gcacaccacctgcttcrtctgtgcggcctgcaa

SEQ ID NO: 24 - DNA, 33 bp, Homo Sapiens
cacctgcttcatttgcrcagtatgtctctagct

SEQ ID NO: 25 - DNA, 33 bp, Homo Sapiens
ggttccattctgccagygtagcaccagatccat

SEQ ID NO: 26 - DNA, 33 bp, Homo Sapiens
cacatccacaggttccrtctaaagccaaaataa

SEQ ID NO: 27 - DNA, 33 bp, Homo Sapiens
actttcagtaccatcaygttggctgaaggagtc

SEQ ID NO: 28 - DNA, 33 bp, Homo Sapiens
tcttctgcatggtagayggtcttttcacaggct

SEQ ID NO: 29 - DNA, 33 bp, Homo Sapiens
attctggctgaagacgyggaagtagtagccatt

SEQ ID NO: 30 - DNA, 33 bp, Homo Sapiens
acgcactggtaggctgygtcgtccgccaatgag

SEQ ID NO: 31 - DNA, 33 bp, Homo Sapiens
cgtaggatctcccacaygtcctcctctgctggt

SEQ ID NO: 32 - DNA, 33 bp, Homo Sapiens
gccactcacgtgcggcrgaaggctgataggagg

SEQ ID NO: 33 - DNA, 33 bp, Homo Sapiens
cacttgaccacaggcgrcttcaggaggctggcg

SEQ ID NO: 34 - DNA, 33 bp, Homo Sapiens
ttgttgcgccgctccaygatggcgatgttctcc

SEQ ID NO: 35 - DNA, 33 bp, Homo Sapiens
ctctgccggaagtcccygtagaggatgcggttg

SEQ ID NO: 36 - DNA, 33 bp, Homo Sapiens
ttctctcagctttcagmtggaagatcacccggg

SEQ ID NO: 37 - DNA, 33 bp, Homo Sapiens
ctcgtgctgcaccttgyggaacttggacaggtt

SEQ ID NO: 38 - DNA, 33 bp, Homo Sapiens
tcctctgccatcatggyggcctgtgtgcaggag

SEQ ID NO: 39 - DNA, 33 bp, Homo Sapiens
gaactccagctgggccyggaggatcttgccctc

SEQ ID NO: 40 - DNA, 33 bp, Homo Sapiens
cccgctgcaggttgtcsatctgctcgcccagct

SEQ ID NO: 41 - DNA, 33 bp, Homo Sapiens
ctccagatcatcgatgwcccttttgagctctga

SEQ ID NO: 42 - DNA, 33 bp, Homo Sapiens
gcctgtcagcttatacrtggagtttttctcctc

SEQ ID NO: 43 - DNA, 33 bp, Homo Sapiens
ctcctcagcgtcatcartggaggccacggtggt

SEQ ID NO: 44 - DNA, 33 bp, Homo Sapiens
gtagggggcggcagccscaaagactgccatctc

SEQ ID NO: 45 - DNA, 33 bp, Homo Sapiens
aggaattacgaatagcwcagacatcacaggctc

SEQ ID NO: 46 - DNA, 33 bp, Homo Sapiens
cttctgctacgagaacragctggccttcctgag

SEQ ID NO: 47 - DNA, 33 bp, Homo Sapiens
ggctcaagatctggatygcatccgactctccac

SEQ ID NO: 48 - DNA, 33 bp, Homo Sapiens
caacttggctcacatcrtgtgagtatccctacc

SEQ ID NO: 49 - DNA, 33 bp, Homo Sapiens
gggccggggcgcgcggrgacggcgcgaggtcgg

SEQ ID NO: 50 - DNA, 33 bp, Homo Sapiens
agcagaagccttcatamaagtaagaatatgctt

SEQ ID NO: 51 - DNA, 33 bp, Homo Sapiens
tgtacgtccatgatgawcagggttgtcaggtca

SEQ ID NO: 52 - DNA, 33 bp, Homo Sapiens
tgtcacttctctttgtygccttgatttctttct

SEQ ID NO: 53 - DNA, 33 bp, Homo Sapiens
ttttcctcctttgacakgaagtcaagttcgctt

SEQ ID NO: 54 - DNA, 33 bp, Homo Sapiens
ctctgttcttttcattrctaagtagtcatcaat

SEQ ID NO: 55 - DNA, 33 bp, Homo Sapiens
aattggtaaagaccctwgtctgactcaaatgtg

SEQ ID NO: 56 - DNA, 33 bp, Homo Sapiens
atgtcaaagtgtccaayattatgactgtgtaaa

SEQ ID NO: 57 - DNA, 33 bp, Homo Sapiens
agcacttgtgttaatgmgctcaaatatgtcaag

SEQ ID NO: 58 - DNA, 33 bp, Homo Sapiens
tgtgactctagaaccaycatcatctttgggccg

SEQ ID NO: 59 - DNA, 33 bp, Homo Sapiens
gtttctgaagtagttcyggtaacattcttcaat

SEQ ID NO: 60 - DNA, 33 bp, Homo Sapiens
tagtctttgttgacacrtgtccagcgcaggctc

SEQ ID NO: 61 - DNA, 33 bp, Homo Sapiens
tagggttcttcccaackaatagacatggcattg

SEQ ID NO: 62 - DNA, 33 bp, Homo Sapiens
tgacacgacagaccatrttctcttggtggcatc

SEQ ID NO: 63 - DNA, 33 bp, Homo Sapiens
gtttacagcagataccyggaagtagtaattgac

SEQ ID NO: 64 - DNA, 33 bp, Homo Sapiens
tctccttgtcttatctygacaacatacccagta

SEQ ID NO: 65 - DNA, 33 bp, Homo Sapiens
tatagatgcgaggtccrtggtattttcaacaca

SEQ ID NO: 66 - DNA, 33 bp, Homo Sapiens
ttcctagcaaagacacrgaattcatactgggaa

SEQ ID NO: 67 - DNA, 33 bp, Homo Sapiens
cctgtaatcttgctacytccatcgaaagctggt

SEQ ID NO: 68 - DNA, 33 bp, Homo Sapiens
cctttcaattatatatmcagatatttcactacc

SEQ ID NO: 69 - DNA, 33 bp, Homo Sapiens
attaacggccacagacygagtgccggcaacatt

SEQ ID NO: 70 - DNA, 33 bp, Homo Sapiens
tatcttaaggacctctycagctttgacaacaat

SEQ ID NO: 71 - DNA, 33 bp, Homo Sapiens
tctccagctttgacaamaataacgtctcggaac

SEQ ID NO: 72 - DNA, 33 bp, Homo Sapiens
attccttgcaaaaaccyggaattcataacgctg

SEQ ID NO: 73 - DNA, 33 bp, Homo Sapiens
attggaagccaagactyggaagtagtaagaaca

SEQ ID NO: 74 - DNA, 33 bp, Homo Sapiens
ttctcttatggtcaaawtcacaggggcacttgg

SEQ ID NO: 75 - DNA, 33 bp, Homo Sapiens
tagcctttaacaggtgygccaccatcataaatt

SEQ ID NO: 76 - DNA, 33 bp, Homo Sapiens
ttgtcctccatcagtcygtatacagtctttgac

SEQ ID NO: 77 - DNA, 33 bp, Homo Sapiens
tctgacactttgctagscttgccaatgccaaca

SEQ ID NO: 78 - DNA, 33 bp, Homo Sapiens
atttgttgacagccatyatacggaaaacatatt

SEQ ID NO: 79 - DNA, 33 bp, Homo Sapiens
tatagtaacaactttgygcaggtcagcatccag

SEQ ID NO: 80 - DNA, 33 bp, Homo Sapiens
ataggtttattccaagygattgaaatggatgat

SEQ ID NO: 81 - DNA, 33 bp, Homo Sapiens
cttgtatccagaacacrtgggttacctggtggt

SEQ ID NO: 82 - DNA, 33 bp, Homo Sapiens
acagtgtcacaagcctygtaaaatggactggta

SEQ ID NO: 83 - DNA, 33 bp, Homo Sapiens
aatgttgctgaagttgrccttcagccaccgtcc

SEQ ID NO: 84 - DNA, 33 bp, Homo Sapiens
aagttggccttcagccrccgtccattaggaagg

SEQ ID NO: 85 - DNA, 33 bp, Homo Sapiens
ccccagtatattcaggsttagcccatttaagtg

SEQ ID NO: 86 - DNA, 33 bp, Homo Sapiens
atttagaggtactggtwttccaggtgggtcaat

SEQ ID NO: 87 - DNA, 33 bp, Homo Sapiens
ttttccaggtgggtcartgggatccagagccaa

SEQ ID NO: 88 - DNA, 33 bp, Homo Sapiens
cttgctgccaccatcgygtttgggcttaggcca

SEQ ID NO: 89 - DNA, 33 bp, Homo Sapiens
gtttacagctgagaccyggaagatgtactcatt

SEQ ID NO: 90 - DNA, 33 bp, Homo Sapiens
ctctaaaggtggttttstggacagctgaggcgc

SEQ ID NO: 91 - DNA, 33 bp, Homo Sapiens
aggttttctgttgaccktagtccagttaacagc

SEQ ID NO: 92 - DNA, 33 bp, Homo Sapiens
tttcagcacaaatacgsaactgatactcatggc

SEQ ID NO: 93 - DNA, 33 bp, Homo Sapiens
atcagaaggttcagaargtgggctaatgtttac

SEQ ID NO: 94 - DNA, 33 bp, Homo Sapiens
tgggctaatgtttaccrcggtcctggcaatagc

SEQ ID NO: 95 - DNA, 33 bp, Homo Sapiens
ctttctgtctgcctcaygtttctccacgatata

SEQ ID NO: 96 - DNA, 33 bp, Homo Sapiens
ttttctgccttgacacrgaactgatattcatgg

SEQ ID NO: 97 - DNA, 33 bp, Homo Sapiens
atacttggttttggctrtgactggtttactttc

SEQ ID NO: 98 - DNA, 33 bp, Homo Sapiens
cacaaaactgccagccrtgttagttgccgtaac

SEQ ID NO: 99 - DNA, 33 bp, Homo Sapiens
gtcactggcatccagaygtctttacccacttcc

SEQ ID NO: 100 - DNA, 33 bp, Homo Sapiens
gggtcccatgcaaggcrctcaacaatatagtgg

SEQ ID NO: 101 - DNA, 33 bp, Homo Sapiens
tcttggtggggatgggyggtctggaaaggaatc

SEQ ID NO: 102 - DNA, 33 bp, Homo Sapiens
attgacagctttgacayggaactcatacatttg

SEQ ID NO: 103 - DNA, 33 bp, Homo Sapiens
tggtaacatcttccacmatgggcttatctggtg

SEQ ID NO: 104 - DNA, 33 bp, Homo Sapiens
aggtgatccagaaatamttgcatcaagtgctat

SEQ ID NO: 105 - DNA, 33 bp, Homo Sapiens
tgctatttcatcacctygtttcacttctaggct

SEQ ID NO: 106 - DNA, 33 bp, Homo Sapiens
gtaatggtataaattcyggcatctgcacggaca

SEQ ID NO: 107 - DNA, 33 bp, Homo Sapiens
aatggtataaattccgrcatctgcacggacact

SEQ ID NO: 108 - DNA, 33 bp, Homo Sapiens
tttatgccaactaacarttggaactgggacagc

SEQ ID NO: 109 - DNA, 33 bp, Homo Sapiens
catcatctggctcacamcatgtgagagtcactg

SEQ ID NO: 110 - DNA, 33 bp, Homo Sapiens
gggccacatttgttacragcacaaactttaaat

SEQ ID NO: 111 - DNA, 33 bp, Homo Sapiens
tttgaactatcaaatakagcttcttcatttctg

SEQ ID NO: 112 - DNA, 33 bp, Homo Sapiens
tatagcttcttcatttytgaaccatttggcttt

SEQ ID NO: 113 - DNA, 33 bp, Homo Sapiens
aatatacaatacttacrcttaactcggaggtgg

SEQ ID NO: 114 - DNA, 33 bp, Homo Sapiens
tctactctaatttgggwggtgtcatcaatagac

SEQ ID NO: 115 - DNA, 33 bp, Homo Sapiens
atagaccccttcatcawcaaattgagaatcatt

SEQ ID NO: 116 - DNA, 33 bp, Homo Sapiens
gaggacacattcgaatygagcctgtcgcctttc

SEQ ID NO: 117 - DNA, 33 bp, Homo Sapiens
ggaggcactggcacttycttttcaggaacaact

SEQ ID NO: 118 - DNA, 33 bp, Homo Sapiens
tcttcaacttcctctaygctaggtggttcttct

SEQ ID NO: 119 - DNA, 33 bp, Homo Sapiens
ctcatattcttcttccsgttgtactgaaacagc

SEQ ID NO: 120 - DNA, 33 bp, Homo Sapiens
tctgggacgggtttctyaggcagagctggcact

SEQ ID NO: 121 - DNA, 33 bp, Homo Sapiens
aacacaaagatgtatasctttcacttcaataac

SEQ ID NO: 122 - DNA, 33 bp, Homo Sapiens
ggtgctacattgatgaycttaaggccggatact

SEQ ID NO: 123 - DNA, 33 bp, Homo Sapiens
actgagttcaggggtgmcagctactgtacactc

SEQ ID NO: 124 - DNA, 33 bp, Homo Sapiens
ttgatttctggagaccyaccgattttgcattca

SEQ ID NO: 125 - DNA, 33 bp, Homo Sapiens
cttcttaatgaacctgkgtggttctatggaacc

SEQ ID NO: 126 - DNA, 33 bp, Homo Sapiens
agaaccccatccttgtmccaagacacttgaaga

SEQ ID NO: 127 - DNA, 33 bp, Homo Sapiens
ttctgagccattgatgyggcattcaaatgcaac

SEQ ID NO: 128 - DNA, 33 bp, Homo Sapiens
tctttcagttttcttgmaaagaaaggtggaagt

SEQ ID NO: 129 - DNA, 33 bp, Homo Sapiens
tactgcatctctcagaktgtgaaataagatact

SEQ ID NO: 130 - DNA, 33 bp, Homo Sapiens
  misc_feature (17)..(17): n is a, c, g, or t
tcagccaatcttttcanaaaggtggctggttct

SEQ ID NO: 131 - DNA, 33 bp, Homo Sapiens
aaaaagatgtgtggtayaggaagcactgccagc

SEQ ID NO: 132 - DNA, 33 bp, Homo Sapiens
gcatgactccccaggcrccagttccctgctgcc

SEQ ID NO: 133 - DNA, 33 bp, Homo Sapiens
attggcagccacacacrtgtatatgcctgtgtc

SEQ ID NO: 134 - DNA, 33 bp, Homo Sapiens
cactgtttttcacttcwaagctatataatcctt

SEQ ID NO: 135 - DNA, 33 bp, Homo Sapiens
catatatattttccagrattagatgcttctgga

SEQ ID NO: 136 - DNA, 33 bp, Homo Sapiens
gccctccactatggccygtaactcaacagcatc

SEQ ID NO: 137 - DNA, 33 bp, Homo Sapiens
ctttctccagcaataayatctatagatacaggc

SEQ ID NO: 138 - DNA, 33 bp, Homo Sapiens
  misc_feature (17)..(17): n is a, c, g, or t
aaacctttcacaaaganacgggtagtgcaagat

SEQ ID NO: 139 - DNA, 33 bp, Homo Sapiens
aatgaaacatttaggayctgagacaagttccac

SEQ ID NO: 140 - DNA, 33 bp, Homo Sapiens
gtcacacttggttatayggaggttaaacacaga

SEQ ID NO: 141 - DNA, 33 bp, Homo Sapiens
gcagctgtgcctccccrgagggagctggtactc

SEQ ID NO: 142 - DNA, 33 bp, Homo Sapiens
ctttaagacttcaattrgcttaaattccttggt

SEQ ID NO: 143 - DNA, 33 bp, Homo Sapiens
ctgagccaggcagaacmtcctttgagccgggtt

SEQ ID NO: 144 - DNA, 33 bp, Homo Sapiens
ggtttagttacaaaacygggtggttctgaagaa

SEQ ID NO: 145 - DNA, 33 bp, Homo Sapiens
gcagcttgcagggtaayggtttgtcctcctagt

SEQ ID NO: 146 - DNA, 33 bp, Homo Sapiens
acaacacattcatattyaccaacatctgcacta

SEQ ID NO: 147 - DNA, 33 bp, Homo Sapiens
ggatccaccttcttcaygaaggatggtggctct

SEQ ID NO: 148 - DNA, 33 bp, Homo Sapiens
acataggttcctgaatstttcagtttggccaaa

SEQ ID NO: 149 - DNA, 33 bp, Homo Sapiens
tttttgcccatctttgytccacgtaactgtgac

SEQ ID NO: 150 - DNA, 33 bp, Homo Sapiens
ggaagctttgcatgtayactcgccgcagtcaac

SEQ ID NO: 151 - DNA, 33 bp, Homo Sapiens
tccatatttggatctasaaaattaaatggaaga

SEQ ID NO: 152 - DNA, 33 bp, Homo Sapiens
aattctatgcttcaccyttttccccgggtagtg

SEQ ID NO: 153 - DNA, 33 bp, Homo Sapiens
ttaaagggagagccagwaaacctcaggtcaacc

SEQ ID NO: 154 - DNA, 33 bp, Homo Sapiens
ctcagcttttataatcygacgaagacctgttgg

SEQ ID NO: 155 - DNA, 33 bp, Homo Sapiens
agctaaaaatcaattarccaccttctacactta

SEQ ID NO: 156 - DNA, 33 bp, Homo Sapiens
ccaggttacatcaatgraagaatcatcttttaa

SEQ ID NO: 157 - DNA, 33 bp, Homo Sapiens
acccggcattgaaaacrggctggctgcccttca

SEQ ID NO: 158 - DNA, 33 bp, Homo Sapiens
aacttggggcggttcargagctaggagtaaatg

SEQ ID NO: 159 - DNA, 33 bp, Homo Sapiens
ttccacactttccaagragactttaacttcaag

SEQ ID NO: 160 - DNA, 33 bp, Homo Sapiens
taggatagcaatggggygggctgtgaaatatgg

SEQ ID NO: 161 - DNA, 33 bp, Homo Sapiens
tccacgacgagatgtarttgtatatttgccatt

SEQ ID NO: 162 - DNA, 33 bp, Homo Sapiens
ccattggatttaagctycacatcattatgatac

SEQ ID NO: 163 - DNA, 33 bp, Homo Sapiens
gaaccttttgcttttgyggatgagctgtccatt

SEQ ID NO: 164 - DNA, 33 bp, Homo Sapiens
gcctgttaccctgcagyggaaccttgcagtctc

SEQ ID NO: 165 - DNA, 33 bp, Homo Sapiens
tcatgaaaccagaacgyctctggcataggtcta

SEQ ID NO: 166 - DNA, 33 bp, Homo Sapiens
ctcatctgtctcctccrgcctacgtccagggga

SEQ ID NO: 167 - DNA, 33 bp, Homo Sapiens
aagaacaacaggtataygcagactagctctgcc

SEQ ID NO: 168 - DNA, 33 bp, Homo Sapiens
ttctccatgcttattgmgaacaacaatagtgta

SEQ ID NO: 169 - DNA, 33 bp, Homo Sapiens
aagcgctcttcacggayggtggtgccagtgatg

SEQ ID NO: 170 - DNA, 33 bp, Homo Sapiens
tatccatgttctgtctyaggaacagaaattttt

SEQ ID NO: 171 - DNA, 33 bp, Homo Sapiens
gtcctgatctgagtagrggttgtcagcgttgtc

SEQ ID NO: 172 - DNA, 33 bp, Homo Sapiens
cctgggaggtgttttaygtggcagctgttgccc

SEQ ID NO: 173 - DNA, 33 bp, Homo Sapiens
agtagctcttccaacgytattggtggcatttac

SEQ ID NO: 174 - DNA, 33 bp, Homo Sapiens
acggagtgcacgcagcrtccgcagtgacttgat

SEQ ID NO: 175 - DNA, 33 bp, Homo Sapiens
ggcctcggcctcagagkaggcagtcgctgacac

SEQ ID NO: 176 - DNA, 33 bp, Homo Sapiens
ttggtgccgttgagcgmtgtgaagttgcgcacg

SEQ ID NO: 177 - DNA, 33 bp, Homo Sapiens
gaactggccatcattcrtggtcagatggcttat

SEQ ID NO: 178 - DNA, 33 bp, Homo Sapiens
tggagcatccggtacasgtccgactcggggggc

SEQ ID NO: 179 - DNA, 33 bp, Homo Sapiens
gaacaagagggcatctrctaaagtttcagattc

SEQ ID NO: 180 - DNA, 33 bp, Homo Sapiens
ccagatttctgctcagyatccagtagtggatca

SEQ ID NO: 181 - DNA, 33 bp, Homo Sapiens
gccaccgtcccgccagscattcgctcctactga

SEQ ID NO: 182 - DNA, 33 bp, Homo Sapiens
cagggccatgacaatcrccaaggtatgtcctca

SEQ ID NO: 183 - DNA, 33 bp, Homo Sapiens
tccaacacagtcatgcrgtttttgaatgagcag

SEQ ID NO: 184 - DNA, 33 bp, Homo Sapiens
cacccggaagagagaayatgaaaatgagctggc

SEQ ID NO: 185 - DNA, 33 bp, Homo Sapiens
gaaattgagaggctgcrgtctctcacagagaac

SEQ ID NO: 186 - DNA, 33 bp, Homo Sapiens
caggagttgatgcttayggcgccgctgaccagg

SEQ ID NO: 187 - DNA, 33 bp, Homo Sapiens
gcatcattaatttgagmtttctttaacgtatct

SEQ ID NO: 188 - DNA, 33 bp, Homo Sapiens
gcaggccaggaactgaygggcttggagtccagg

SEQ ID NO: 189 - DNA, 33 bp, Homo Sapiens
ctcagagacgtgaaggyctttaagtcatccata

SEQ ID NO: 190 - DNA, 33 bp, Homo Sapiens
acacagtatcctgagcygtccaaacactcgttg

SEQ ID NO: 191 - DNA, 33 bp, Homo Sapiens
cacgaccaatgcaacakgtggagaaaaaggacc

SEQ ID NO: 192 - DNA, 33 bp, Homo Sapiens
taggtgttccagatcgygtatgtgattgtgaag

SEQ ID NO: 193 - DNA, 33 bp, Homo Sapiens
acaaagacccaagagamattgaccccattgtca

SEQ ID NO: 194 - DNA, 33 bp, Homo Sapiens
tgatggatgccctgtcrggtacacaggaccacg

SEQ ID NO: 195 - DNA, 33 bp, Homo Sapiens
aggccaatgcaactgtyatccaaaattctctgg

SEQ ID NO: 196 - DNA, 33 bp, Homo Sapiens
agatctccatttggaasctttttattggaaact

SEQ ID NO: 197 - DNA, 33 bp, Homo Sapiens
ttgccgggattttatcsactgcgttctcaacca

SEQ ID NO: 198 - DNA, 33 bp, Homo Sapiens
gggaaggaagtgtgacrgctgcaagcactggca

SEQ ID NO: 199 - DNA, 33 bp, Homo Sapiens
tcattaggctaccaaartgacagcagatggcga

SEQ ID NO: 200 - DNA, 33 bp, Homo Sapiens
tgtggatgcaaatgcartgctgtttgttggtgg

SEQ ID NO: 201 - DNA, 33 bp, Homo Sapiens
tttgatgacaccaaagktaaaaaccggtatgta

SEQ ID NO: 202 - DNA, 33 bp, Homo Sapiens
atccccaccaaaatcartgatggccagtggcac

SEQ ID NO: 203 - DNA, 33 bp, Homo Sapiens
ttccgcacaactacaamgactggagttcttctg

SEQ ID NO: 204 - DNA, 33 bp, Homo Sapiens
gaaaactattgagaagktaaatgagtttcaatt

SEQ ID NO: 205 - DNA, 33 bp, Homo Sapiens
gtctaataacttctccrgttccttgatatgacg

SEQ ID NO: 206 - DNA, 33 bp, Homo Sapiens
ctgaagtatctctgcakcaaggttagaatcaat

SEQ ID NO: 207 - DNA, 33 bp, Homo Sapiens
tccgagttacaggaatygtagactattggcttg

SEQ ID NO: 208 - DNA, 33 bp, Homo Sapiens
agagtatctggcctccrtgaggtaactgtttat

SEQ ID NO: 209 - DNA, 33 bp, Homo Sapiens
attcagaccactcctcygggagccaatgatcat

SEQ ID NO: 210 - DNA, 33 bp, Homo Sapiens
gccacagctcgaaggcrtgtccagcgctgccag

SEQ ID NO: 211 - DNA, 33 bp, Homo Sapiens
tgcagcgtaagtagaakattttcatattctgga

SEQ ID NO: 212 - DNA, 33 bp, Homo Sapiens
agcgtaagtagaagatkttcatattctggagat

SEQ ID NO: 213 - DNA, 33 bp, Homo Sapiens
tgttcaggcgattcctycttctttgttaactta

SEQ ID NO: 214 - DNA, 33 bp, Homo Sapiens
gattgtctccaggctgsttgtctccaggaggtc

SEQ ID NO: 215 - DNA, 33 bp, Homo Sapiens
cactatctcctcatccrtgttcttgccttccag

SEQ ID NO: 216 - DNA, 33 bp, Homo Sapiens
tggtcaagcaggacgawcttctctttcagacct

SEQ ID NO: 217 - DNA, 33 bp, Homo Sapiens
ttgtgtactttgagccrtgaaatccttcagggt

SEQ ID NO: 218 - DNA, 33 bp, Homo Sapiens
actactgtgaattttcytcagctcagataacaa

SEQ ID NO: 219 - DNA, 33 bp, Homo Sapiens
atagcgatcccagttaragatcacttcttccag

SEQ ID NO: 220 - DNA, 33 bp, Homo Sapiens
aggtgtagctctgatgyggaggaaacaaaatga

SEQ ID NO: 221 - DNA, 33 bp, Homo Sapiens
gtgtagctctgatgtgraggaaacaaaatgaaa

SEQ ID NO: 222 - DNA, 33 bp, Homo Sapiens
tcggatgcacccccctwaaaaagtggactcagc

SEQ ID NO: 223 - DNA, 33 bp, Homo Sapiens
cccactctgcggccgcygtggcctgcgcaccga

SEQ ID NO: 224 - DNA, 33 bp, Homo Sapiens
catcctggcttccaggyggcctttgtgttgacg

SEQ ID NO: 225 - DNA, 33 bp, Homo Sapiens
gttccacactctttgtktccaatgcaggcaagt

SEQ ID NO: 226 - DNA, 33 bp, Homo Sapiens
acccatgtcctgatggyactcatggtctcctga

SEQ ID NO: 227 - DNA, 33 bp, Homo Sapiens
tgttcttcagtaagacrttgccatttgagaagg

SEQ ID NO: 228 - DNA, 33 bp, Homo Sapiens
cattagaaatctctccktgtgcttgcaatgtgt

SEQ ID NO: 229 - DNA, 33 bp, Homo Sapiens
tcctggatggcttcaaygctcacttgttgaggc

SEQ ID NO: 230 - DNA, 33 bp, Homo Sapiens
cgacctacgttctgttygcctgaggtcggtcag

SEQ ID NO: 231 - DNA, 33 bp, Homo Sapiens
cttcccaagaaagaggrcgctttactctaccag

(19) United States
(12) Patent Application Publication
(10) Pub. No.: US 2019/0002981 A1
(43) Pub. Date: Jan. 3, 2019

(54) Method of Testing for Preeclampsia and Treatment Therefor

(71) Applicant: Taueret Laboratories, LLC, Salt Lake City, UT (US)

(72) Inventors: Kenneth Ward, Salt Lake City, UT (US); Rakesh N. Chettier, West Jordan, UT (US)

(73) Assignee: Taueret Laboratories, LLC, Salt Lake City, UT (US)

(21) Appl. No.: 15/922,648

(22) Filed: Mar. 15, 2018

Related U.S. Application Data
(60) Provisional application No. 62/471,849, filed on Mar. 15, 2017.
(60) Provisional application No. 62/471,851, filed on Mar. 15, 2017.
(60) Provisional application No. 62/471,857, filed on Mar. 15, 2017.

(51) Int. Cl. C12Q 1/6883; G16H 50/30

(52) U.S. Cl. CPC C12Q 1/6883; G16H 50/30; C12Q 2600/158; C12Q 2600/106; C12Q 2600/124; C12Q 2600/118; C12Q 2600/172

(57) ABSTRACT

The present invention relates to novel genetic markers associated with preeclampsia and risk of developing preeclampsia, and methods and materials for determining whether a human subject has preeclampsia or is at risk of developing preeclampsia and the use of such risk information in selectively administering a treatment that at least partially prevents or compensates for an preeclampsia related condition.

### CROSS REFERENCE TO RELATED APPLICATIONS

This nonprovisional utility patent application claims the benefit under 35 USC § 119(e) of U.S. provisional application Nos. 62/471,849, 62/471,851, and 62/471,857, all filed Mar. 15, 2017, and all of which are incorporated, in their entirety, by this reference.

### FIELD OF THE INVENTION

The present invention relates to prognosis and diagnosis of preeclampsia and to treating preeclampsia, and more especially to treating preeclampsia in subjects having at least one genetic mutation associated with preeclampsia. In particular, the present invention relates to a novel algorithmic combination of preeclampsia associated single nucleotide polymorphisms (SNPs) and Rare Variants (RVs) such as insertion deletion polymorphisms (indels), synonymous variants, nonsynonymous variants, splicing variants, genomic rearrangements, loss of function variants, and preeclampsia related clinical analysis to result in a preeclampsia predictive and/or diagnostic test and related treatment therefor.

### BACKGROUND OF THE INVENTION

Preeclampsia or PE (see Appx A) is a condition that affects a large percentage of women. Family studies have confirmed the heritability of preeclampsia and GWAS (Genome Wide Association Study) studies have implicated several chromosomal regions. Further, genetic mutations associated with preeclampsia have been shown to be useful in predicting existence or predisposition to preeclampsia.

A method of clinically assessing a predisposition to preeclampsia is to determine the existence of at least one preeclampsia associated clinical factor. The results of assessment are compiled into a preeclampsia Raw Clinical Probability Value (RCPV). The RCPV is preferably multiplied by a Relevance Factor (RF) based on a patient's age and race to result in a Final Clinical Probability Value (FCPV). Alternatively, the RCPV may be used with data collected in population surveys.

MultiDimensional Analysis (MDA) is an analysis process that groups data into two or more categories (e.g. cases and controls or patients having a high probability of preeclampsia and patients having a low probability of preeclampsia).

Logistic regression analysis is a process that is used for prediction of the probability of occurrence of an event by fitting data to a logit function logistic curve.

Bayesian analysis or Bayesian interference is a method of statistical inference in which evidence is used to estimate parameters and predictions in a probability model.

Various genetic markers are known to have a predictive association with various female reproductive related conditions. Such genetic markers and methods of detection and use in treatment are disclosed for instance in US patents and application U.S. Pat. Nos. 8,932,993, 9,434,991, 9,840,738, and 2016/0367568, all of which are incorporated herein in their entirety by this reference.

### SUMMARY OF THE INVENTION

The present invention is a method of treating a patient having at least one genetic mutation associated with preeclampsia such that the patient is prevented from developing preeclampsia or such that preeclampsia in the patient is prevented from progressing. More specifically, the invention defines a method for preeclampsia diagnosis/prognosis that preferably combines known preeclampsia clinical factor assessment methods with preeclampsia associated biomarkers such as single nucleotide polymorphisms (SNPs), indels, insertions, deletions, genomic rearrangements, Rare Variants (RVs), and more especially the biomarkers identified in table 1 (or diagnostically and predicatively functionally comparable biomarkers), preferably via a statistical assessment method such as MultiDimensional Scaling analysis (MDS), logistic regression, or Bayesian analysis. The markers and related statistical data shown in table 1 were discovered by analyzing a number of preeclampsia cases and controls much as has been described in the prior patent applications incorporated herein by reference. It is noted that all of the biomarkers of table 1, being variations or mutations in and of the same structure (i.e. the human genome), share a single structural similarity in that all of the biomarkers of table 1 are preeclampsia associated nucleotide substitutions of the same DNA sequence—the human genome DNA sequence, and that the common use of preeclampsia diagnosis and prognosis of all of the biomarkers of table 1 flow from such single structural similarity. The present invention further preferably includes the treatment of a subject determined to have or be predisposed to preeclampsia by administering to such subject a therapeutic such as an a blood pressure reduction medication, a corticosteroid, an anticonvulsant, magnesium sulfate, earlier delivery of the fetus, or a combination thereof that at least partially compensates for preeclampsia or that prevents or reduces the severity of preeclampsia that the subject would otherwise develop or that prevents preeclampsia related complications or associated disorders. It shall be noted that preventing or cancelling a procedure, especially an invasive procedure, such as surgical delivery of the baby through the mother's abdomen via caesarean section, that would otherwise have been performed on a subject but for the results of a (negative) diagnosis/prognosis disclosed herein being performed on said subject, shall be consider within the scope of treatment or the “administration of a therapeutic”.

It shall be noted that for the purposes of this application, a SNP is understood to be a genetic polymorphism having a Minor Allele Frequency (MAF) of at least 1% in a population (such as for instance the Caucasian population or the CEU population) and an RV is understood to be a genetic polymorphism having a Minor Allele Frequency (MAF) of less than 1% in a population (such as for instance the Caucasian population or the CEU population).

It shall be noted that “Linkage disequilibrium” or “LD” means that a particular combination of alleles (alternative nucleotides) or genetic markers at two or more different SNP (or RV) sites are non-randomly co-inherited (i.e., the combination of alleles at the different SNP (or RV) sites occurs more or less frequently in a population than the separate frequencies of occurrence of each allele or the frequency of a random formation of haplotypes from alleles in a given population). The term “LD” differs from “linkage,” which describes the association of two or more loci on a chromosome with limited recombination between them. LD is also used to refer to any non-random genetic association between allele(s) at two or more different SNP (or RV) sites. Therefore, when a SNP (or RV) is in LD with other SNPs (or RVs), the particular allele of the first SNP (or RV) often predicts which alleles will be present in those SNPs (or RVs) in LD. LD is generally, but not exclusively, due to the physical proximity of the two loci along a chromosome. Hence, genotyping one of the SNP (or RV) sites will give almost the same information as genotyping the other SNP (or RV) site that is in LD. Linkage disequilibrium is caused by fitness interactions between genes or by such non-adaptive processes as population structure, inbreeding, and stochastic effects.

It shall also be noted that LD is the non-random association of alleles adjacent loci. When a particular allele at one locus is found together on the same chromosome with a specific allele at a second locus-more often than expected if the loci were segregating independently in a population-the loci are in disequilibrium. This concept of LD is formalized by one of the earliest measures of disequilibrium to be proposed (symbolized by D). D, in common with most other measures of LD, quantifies disequilibrium as the difference between the observed frequency of a two-locus haplotype and the frequency it would be expected to show if the alleles are segregating at random. A wide variety of statistics have been proposed to measure the amount of LD, and these have different strengths, depending on the context. Although the measure D has the intuitive concepts of LD, its numerical value is of little use for measuring the strength of and comparing levels of LD. This is due to the dependence of D on allele frequencies. The two most common measures are the absolute value of D′ and r². The absolute value of D′ is determined by dividing D by its maximum possible value, given the allele frequencies at the two loci. The case of D′=1 is known as complete LD (or CLD). The measure r² is in some ways complementary to D′. An r² value of 1 indicates complete LD as well while an r² value of 0 indicates linkage equilibrium. Complete LD demonstrates complete dependency. In other words, in complete LD the number of counts of the minor allele in loci 1 corresponds to the counts of minor allele in loci 2. Although in complete LD the alleles themselves might be different the frequency of Minor allele in loci 1 will be equal to the frequency of Minor allele in loci 2. For example, in comparing two loci such as rs1 having (A/G) and rs2 having (G/C), if it is known that rs1 and rs2 are in complete LD, and if it is known that a person carries a genotype AG on rs1, then it is known that the genotype on rs2 is GC for that person. Similarly in complete LD, if A is the minor allele of rs1 and is associated with the disease (or conversely is not associated with the disease) then the corresponding minor allele of rs² is also associated with the disease (or conversely or is not associated with the disease). Furthermore in complete LD, in any analysis of the disease, genotype for rs1 could easily be substituted for rs2 and vice versa.

In yet another embodiment, the invention also provides a kit comprising SNP detection reagents, and methods for detecting the SNPs disclosed herein by employing detection reagents and a questionnaire of non-genetic clinical factors. In one embodiment, the questionnaire would be completed by a medical professional based on medical history physical exam or other clinical findings. In yet another embodiment, the questionnaire would include any other non-genetic clinical factors known to be associated with the risk of developing preeclampsia.

In yet another embodiment, genetic markers are used in the selection of patients to whom a therapeutic will be administered based on the patient's assessed risk of developing preeclampsia or based on the patient's assessed risk of preeclampsia progression. The administered therapeutic may preferably be a patient specific gene or protein based therapy enabled and informed by SNPs discovered to be associated with preeclampsia.

It shall also be noted that unless indicated otherwise, when a genetic marker (e.g. SNP or RV) is identified as the genetic marker associated with a disease (in this instance preeclampsia), it shall be understood that it is the minor allele (MA) of the particular genetic marker that is associated with the disease. Further it shall also be noted that unless indicated otherwise, if the Odds Ratio (OR) of the MA is greater than 1.0, the MA of the genetic marker (in this instance the preeclampsia associated genetic marker) is correlated with an increased risk of preeclampsia in a case subject as compared to a control subject and shall be considered a causative marker (C), and if the OR of the MA less than 1.0, the MA of the genetic marker is correlated with a decreased risk of preeclampsia in a case subject as compared to a control subject and shall be considered a protective marker (P).

It shall also be noted that unless indicated otherwise, the phrase “functional equivalent” as used herein with respect to biomarkers shall mean that a second biomarker is substantially equivalent in its diagnostic and/or prognostic value with respect to a given disease as is a first biomarker's diagnostic and/or prognostic value with respect to the given disease. A second biomarker that is in complete LD with a first biomarker shall be expressly included within the scope of “functional equivalent” with respect to the relationship between the second biomarker to the first biomarker.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention can be practiced without one or more of the specific details, or with other methods, components, materials, and so forth. In other instances, well-known structures, materials, or operations are not shown or described in detail to avoid obscuring aspects of the invention.

The present invention is a method of determining an existence or predisposition to preeclampsia in a patient, including for instance in a preeclampsia asymptomatic patient, by observing at least one of a preeclampsia associated clinical factor and at least one at least one genetic mutation associated with preeclampsia and administering treatment therefor such that the patient is prevented from developing preeclampsia or such that preeclampsia in the patient is prevented from progressing. Exemplary treatments include administering to the patient a therapeutic such as an a blood pressure reduction medication, a corticosteroid, an anticonvulsant, magnesium sulfate, earlier delivery of the fetus (including via caesarean section or “C-section”), or a combination thereof that at least partially compensates for preeclampsia or that prevents or reduces the severity of preeclampsia that the subject would otherwise develop or that prevents preeclampsia related complications or associated disorders. An exemplary method of determining existence or predisposition to preeclampsia for a patient is performed according to the following steps. In a first step, a clinical assessment of the patient is performed resulting in at least one observed preeclampsia associated clinical factor. In a second step, an RCPV according to is determined for the patient based on the answers obtained for the patient in step 1. In an optional third step, the RCPV is optionally multiplied by an RF or otherwise adjusted according to the patient's age and race or according to relevant population survey data to result in a FCPV. In a fourth step, at least one preeclampsia associated biomarker preferably drawn from the biomarkers of table 1 is identified in genetic material of the patient. In a fifth step, at least one statistical analysis (preferably MDS) is performed to combine the RCPV (or the FCPV) and the predictive value of the identified genetic biomarker to result in a highly predictive preeclampsia prognosis or diagnosis.

The present invention provides SNPs associated with preeclampsia, nucleic acid molecules containing SNPs, methods and reagents for the detection of the SNPs disclosed herein, uses of these SNPs for the development of detection reagents, and assays or kits that utilize such reagents. The SNPs disclosed herein are useful for diagnosing, screening for, and evaluating predisposition to preeclampsia and progression of preeclampsia. Additionally, such SNPs are useful in the determining individual subject treatment plans and design of clinical trials of devices for possible use in the treatment of preeclampsia. Furthermore, such SNPs and their encoded products are useful targets for the development of therapeutic agents. Furthermore, such SNPs combined with other non-genetic clinical factors are useful for diagnosing, screening, evaluating predisposition to preeclampsia, assessing risk of progression of preeclampsia, determining individual subject treatment plans and design of clinical trials of devices for possible use in the treatment of preeclampsia. Furthermore, such SNPs and are useful in the selection of recipients for a preeclampsia type therapeutic.

It shall be noted that the markers of table 1 are drawn from build 37 data (or “GRCh37” as defined by the Genome Reference Consortium) and that in the header of table 1: “Chr” corresponds to the chromosome where a given biomarker is located in the human genome, “Gene” corresponds to the gene where a given biomarker is located in the human genome or alternatively if the biomarker is not located within a gene, “Gene” corresponds to the nearest two genes positioned on either side of the given biomarker in the human genome, “position” corresponds to the position of a given biomarker in the human genome, “Amino Acid Position” corresponds to the protein level changes as a result of the DNA level changes, “p-value” corresponds to the p-value of a given biomarker, “OR [L95-U95]” corresponds to a measure of association which compares the odds of disease/condition of those exposed to the odds of disease/condition those unexposed and the L95 value is the lower endpoint and the U95 value is the upper endpoint of the 95% confidence interval, “Case MAF” corresponds to the case Minor Allele Frequency of a given biomarker, “Cont MAF” corresponds to the control Minor Allele Frequency of a given biomarker, “Ref/Alt(MA)” corresponds to the reference allele/alternate allele (Minor allele or MA) of a given biomarker, and “Context Sequence” corresponds to the context sequence in which a given biomarker is located and provides a SEQ ID NO and the identification of the biomarker variation of substitution (e.g. “A/C” or “A/G”, etc.). It shall be further noted that values for p-value, OR, Case MAF, and Cont MAF provided in Table 1 were derived by applicant using predetermined statistical methods and a predetermined group of cases and controls, and that while others who might analyze the same set of data may arrive at similar but not necessarily identical results if the identical analytical methods are not used. Moreover, it is believed that substantially similar results would occur based on a similar analysis performed on data drawn from different populations than that used herein. Some of the variants listed in Table 1 can be splicing variants, for example NM_001256850:exon116:c.30475+1G>C, NM_001267550:exon118:c.31426+1G>C, NM_133378:exon115:c.27694+1G>C. The NM number indicates that a particular GenBank cDNA reference sequence was used for reference. The “c” indicates that the nucleotide number which follows is based on coding DNA sequence. The numbers provide the position of the mutation in the DNA. For instance, 30475+1G>C means one base after (+1) the 30475^{th} coding nucleotide at the end of the exon is mutated form a G to a C.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

Tables

TABLE 1
(Based on build 37 or ″GRCh37″ as defined by the Genome Reference Consortium)

| | | | Amino | | | | | | |
|---|---|---|---|---|---|---|---|---|---|
| | | | Acid | | OR | Case | Cont | Ref/Alt | |
| Chr | Gene | Position | Position | p-value | [L95-U95] | MAF | MAF | (MA) | Context Sequence |
| 1 | KDF1 | 27278439 | p.R145W | 8.42E-09 | 302.28[75.31 - | 0.01105 | 0.00004 | G/A | (SEQ ID NO: 0001) |
| | | | | | 1213.36] | | | | cgctggccatcccgcc[G/A]gctggggggtgcacga |
| 1 | TOE1 | 45806768 | p.S26A | 2.06E-06 | 52[17.95 - | 0.01105 | 0.00021 | T/G | (SEQ ID NO: 0002) |
| | | | | | 150.63] | | | | cagcaaaagcacaac[T/G]ctggggaggagctagt |
| 1 | NEXN | 78395029 | p.T298R | 6.42E-02 | 15.88[2.12 - | 0.00276 | 0.00017 | C/G | (SEQ ID NO: 0003) |
| | | | | | 118.97] | | | | gaggaaaaccaagaca[C/G]agcaaaaatttttaaa |
| 1 | NEXN | 78399103 | p.R397Q | 6.47E-03 | 308.35[19.25 - | 0.00276 | 0.00001 | G/A | (SEQ ID NO: 0004) |
| | | | | | 4939.19] | | | | cgaacagaggaggaac[G/A]gaagcataagctagaa |
| 1 | TCHHL1 | 152058192 | p.Q656X | 1.73E-11 | 81.56[35.29 - | 0.00000 | 0.00024 | G/A | (SEQ ID NO: 0005) |
| | | | | | 188.51] | | | | cctgctgtggattcct[G/A]tgcttctgggtgtcca |
| 1 | LMNA | 156105759 | p.R335Q | 1.93E-02 | 61.63[7.18 - | 0.00276 | 0.00004 | G/A | (SEQ ID NO: 0006) |
| | | | | | 528.82] | | | | gagcgggacaccagcc[G/A]gcggctgctggcggaa |
| 1 | LMNA | 156107469 | p.R545C | 5.93E-03 | Inf | 0.00276 | 0.00000 | C/T | (SEQ ID NO: 0007) |
| | | | | | | | | | catgcgcaagctggtg[C/T]gctcagtgactgtggt |
| 1 | TNNT2 | 201330455 | p.E244D | 1.68E-01 | 5.52[0.76 - | 0.00276 | 0.00050 | C/A | (SEQ ID NO: 0008) |
| | | | | | 40.01] | | | | ggtcgaacttctctgc[C/A]tccaagttatagatgc |
| 1 | TNNT2 | 201331068 | p.I221T | 7.47E-02 | 13.45[1.81 - | 0.00276 | 0.00021 | A/G | (SEQ ID NO: 0008) |
| | | | | | 99.88] | | | | ttcattcaggtggtca[A/G]tggccagcaccttcct |
| 1 | TNNT2 | 201332480 | p.A1725 | 6.45E-03 | 309.47[19.32 - | 0.00276 | 0.00001 | C/A | (SEQ ID NO: 0010) |
| | | | | | 4957.2] | | | | gccttcttcttccggg[C/A]ctcatcctcagccttc |
| 1 | TNNT2 | 201338944 | p.E33X | 6.45E-03 | 309.44[19.32 - | 0.00276 | 0.00001 | C/A | (SEQ ID NO: 0011) |
| | | | | | 4956.7] | | | | ggaaaggctgtactac[C/A]gtcttcgtcctctctc |
| 1 | ACTN2 | 236894607 | p.D230E | 6.57E-02 | 15.46[2.07 - | 0.00276 | 0.00018 | T/A | (SEQ ID NO: 0012) |
| | | | | | 115.47] | | | | ttcctaaaatgttgga[T/A]gctgaaggtgagatga |
| 1 | ACTN2 | 236906260 | p.N391S | 3.23E-03 | Inf | 0.00276 | 0.00000 | A/G | (SEQ ID NO: 0013) |
| | | | | | | | | | gaggagtggttgctca[A/G]tgagattcggagactg |
| 1 | ACTN2 | 236910983 | p.D475N | 9.66E-03 | 154.7[14 - | 0.00276 | 0.00002 | G/A | (SEQ ID NO: 0014) |
| | | | | | 1709.87] | | | | tgaactggactatcac[G/A]acgctgtgaatgtcaa |
| 10 | VCL | 75849841 | p.A413T | 1.48E-02 | 11.27[2.74 - | 0.00552 | 0.00049 | G/A | (SEQ ID NO: 0015) |
| | | | | | 46.38] | | | | gattcgaggtgctttg[G/A]ctgaagctcggaaaat |
| 10 | VCL | 75855510 | p.R547Q | 9.66E-03 | 154.69[14 - | 0.00276 | 0.00002 | G/A | (SEQ ID NO: 0016) |
| | | | | | 1709.78] | | | | ctcgccaagtgtgacc[G/A]agtggaccagctgaca |
| 10 | LDB3 | 88451789 | p.R344C | 3.23E-03 | Inf | 0.00276 | 0.00000 | C/T | (SEQ ID NO: 0017) |
| | | | | | | | | | gcagtctcgctccttc[C/T]gcatcctggcccagat |
| 10 | LDB3 | 88466346 | p.A319T | 1.94E-02 | 61.29[7.14 - | 0.00276 | 0.00005 | G/A | (SEQ ID NO: 0018) |
| | | | | | 525.94] | | | | caccccgctgctgccc[G/A]cttctgcccagccacc |
| 10 | LDB3 | 88469762 | p.R401G | 3.28E-03 | Inf | 0.00276 | not seen | C/G | (SEQ ID NO: 0019) |
| | | | | | | | | | ccctgcacccaagccc[C/G]gggttgtcaccactgc |
| 10 | LDB3 | 88476105 | p.P423R | 6.43E-02 | 15.86[2.12 - | 0.00276 | 0.00017 | C/G | (SEQ ID NO: 0020) |
| | | | | | 118.78] | | | | gcatctacctacagcc[C/G]gtccccaggggccaat |
| 10 | LDB3 | 88476287 | p.G484R | 4.20E-02 | 25.26[3.28 - | 0.00276 | 0.00011 | G/A | (SEQ ID NO: 0021) |
| | | | | | 194.77] | | | | ctcggtggcctacagc[G/A]ggggccctgcggagcc |
| 10 | LDB3 | 88476458 | p.V541I | 1.61E-02 | 76.88[8.57 - | 0.00276 | 0.00004 | G/A | (SEQ ID NO: 0022) |
| | | | | | 689.53] | | | | acttgccagggggacc[G/A]tccagagggctgagcg |
| 10 | LDB3 | 88478529 | p.V640I | 1.71E-01 | 5.43[0.75 - | 0.00276 | 0.00051 | G/A | (SEQ ID NO: 0023) |
| | | | | | 39.29] | | | | gcacaccacctgcttc[G/A]tctgtgcggcctgcaa |
| 10 | LDB3 | 88486007 | p.A703T | 1.55E-01 | 6.06[0.84 - | 0.00276 | 0.00046 | G/A | (SEQ ID NO: 0024) |
| | | | | | 43.97] | | | | cacctgcttcatttgc[G/A]cagtatgtctctagct |
| 10 | ANKRD1 | 92672702 | p.H294R | 1.96E-02 | 60.72[7.08 - | 0.00276 | 0.00005 | T/C | (SEQ ID NO: 0025) |
| | | | | | 521] | | | | ggttccattctgccag[T/C]gtagcaccagatccat |
| 10 | ANKRD1 | 92678728 | p.T116M | 9.07E-02 | 10.9[1.48 - | 0.00276 | 0.00025 | G/A | (SEQ ID NO: 0026) |
| | | | | | 80.34] | | | | cacatccacaggttcc[G/A]tctaaagccaaaataa |
| 10 | ANKRD1 | 92680784 | p.M1V | 6.48E-03 | 308.15[19.24 - | 0.00276 | 0.00001 | T/C | (SEQ ID NO: 0027) |
| | | | | | 4936.08] | | | | actttcagtaccatca[T/C]gttggctgaaggagtc |
| 11 | CSRP3 | 19213947 | p.V17I | 6.45E-03 | 309.32[19.31 - | 0.00276 | 0.00001 | C/T | (SEQ ID NO: 0028) |
| | | | | | 4954.81] | | | | (SEQ ID NO: 0029) |
| | | | | | | | | | tcttctgcatggtaga[C/T]ggtcttttcacaggct |
| 11 | MYBPC3 | 47354442 | p.R1138H | 1.12E-01 | 8.66[1.18 - | 0.00276 | 0.00032 | C/T | (SEQ ID NO: 0030) |
| | | | | | 63.46] | | | | attctggctgaagacg[C/T]ggaagtagtagccatt |
| 11 | MYBPC3 | 47364629 | p.A432T | 1.64E-02 | 75.56[8.42 - | 0.00276 | 0.00004 | C/T | (SEQ ID NO: 0031) |
| | | | | | 677.65] | | | | acgcactggtaggctg[C/T]gtcgtccgccaatgag |
| 11 | MYBPC3 | 47367887 | p.V321M | 1.90E-01 | 4.83[0.67 - | 0.00276 | 0.00057 | C/T | (SEQ ID NO: 0032) |
| | | | | | 34.91] | | | | cgtaggatctcccaca[C/T]gtcctcctctgctggt |
| 11 | MYBPC3 | 47369415 | p.R272C | 3.94E-02 | 28.43[3.49 - | 0.00276 | 0.00010 | G/A | (SEQ ID NO: 0033) |
| | | | | | 231.65] | | | | gccactcacgtgcggc[G/A]gaaggctgataggagg |
| 11 | MYBPC3 | 47371423 | p.P186S | 3.28E-03 | Inf | 0.00276 | not seen | G/A | (SEQ ID NO: 0034) |
| | | | | | | | | | cacttgaccacaggcg[G/A]cttcaggaggctggcg |
| 14 | MYH6 | 23855274 | p.V1676M | 9.66E-03 | 154.66[13.99 - | 0.00276 | 0.00002 | C/T | (SEQ ID NO: 0035) |
| | | | | | 1709.47] | | | | ttgttgcgccgctcca[C/T]gatggcgatgttctcc |
| 14 | MYH6 | 23866188 | p.G718R | 3.28E-03 | Inf | 0.00276 | not seen | C/T | (SEQ ID NO: 0036) |
| | | | | | | | | | ctctgccggaagtccc[C/T]gtagaggatgcggttg |
| 14 | MYH6 | 23872624 | p.Q277H | 1.38E-01 | 6.87[0.95 - | 0.00276 | 0.00040 | C/A | (SEQ ID NO: 0037) |
| | | | | | 50] | | | | ttctctcagctttcag[C/A]tggaagatcacccggg |
| 14 | MYH7 | 23883068 | p.R1897H | 3.23E-03 | Inf | 0.00276 | 0.00000 | C/T | (SEQ ID NO: 0038) |
| | | | | | | | | | ctcgtgctgcaccttg[C/T]ggaacttggacaggtt |
| 14 | MYH7 | 23884476 | p.A1763T | 3.81E-02 | 28.13[3.62 - | 0.00276 | 0.00010 | C/T | (SEQ ID NO: 0039) |
| | | | | | 218.46] | | | | tcctctgccatcatgg[C/T]ggcctgtgtgcaggag |
| 14 | MYH7 | 23885487 | p.R1560Q | 3.24E-03 | Inf | 0.00276 | 0.00000 | C/T | (SEQ ID NO: 0040) |
| | | | | | | | | | gaactccagctgggcc[C/T]ggaggatcttgccctc |
| 14 | MYH7 | 23889159 | p.I1207M | 3.28E-03 | Inf | 0.00276 | not seen | G/C | (SEQ ID NO: 0041) |
| | | | | | | | | | cccgctgcaggttgtc[G/C]atctgctcgcccagct |
| 14 | MYH7 | 23893180 | p.D953V | 6.45E-03 | 309.47[19.32 - | 0.00276 | 0.00001 | T/A | (SEQ ID NO: 0042) |
| | | | | | 4957.2] | | | | ctccagatcatcgatg[T/A]cccttttgagctctga |
| 14 | MYH7 | 23899076 | p.M349T | 3.28E-03 | Inf | 0.00276 | not seen | A/G | (SEQ ID NO: 0043) |
| | | | | | | | | | gcctgtcagcttatac[A/G]tggagtttttctcctc |
| 14 | MYH7 | 23899800 | p.I323T | 6.45E-03 | 309.23[19.3 - | 0.00276 | 0.00001 | A/G | (SEQ ID NO: 0044) |
| | | | | | 4953.39] | | | | ctcctcagcgtcatca[A/G]tggaggccacggtggt |
| 14 | MYH7 | 23902913 | p.G10A | 1.93E-02 | 61.76[7.2 - | 0.00276 | 0.00004 | C/G | (SEQ ID NO: 0045) |
| | | | | | 529.97] | | | | gtagggggcggcagcc[C/G]caaagactgccatctc |
| 14 | FAM71D | 67671483 | p.T197S | 3.69E-13 | Inf | 0.01381 | not seen | A/T | (SEQ ID NO: 0046) |
| | | | | | | | | | aggaattacgaatagc[A/T]cagacatcacaggctc |
| 17 | KCNJ18 | 21319792 | p.E380K | 1.01E-10 | 61.3[27.05 - | 0.01934 | 0.00032 | G/A | (SEQ ID NO: 0047) |
| | | | | | 138.93] | | | | cttctgctacgagaac[G/A]agctggccttcctgag |
| 18 | DTNA | 32345942 | p.R29C | 1.29E-02 | 103.02[10.69 - | 0.00276 | 0.00003 | C/T | (SEQ ID NO: 0048) |
| | | | | | 992.71] | | | | ggctcaagatctggat[C/T]gcatccgactctccac |
| 18 | DTNA | 32400878 | p.V16M | 2.36E-01 | 3.77[0.52 - | 0.00276 | 0.00073 | G/A | (SEQ ID NO: 0049) |
| | | | | | 27.15] | | | | caacttggctcacatc[G/A]tgtgagtatccctacc |
| 19 | KANK3 | 8400493 | p.P73L | 7.99E-01 | 1.12[0.41 - | 0.01657 | 0.01482 | G/A | (SEQ ID NO: 0050) |
| | | | | | 3.05] | | | | gggccggggcgcgcgg[G/A]gacggcgcgaggtcgg |
| 2 | NDUFAF7 | 37471114 | p.Q166K | 2.00E-13 | 96.1[43.21 - | 0.02210 | 0.00024 | C/A | (SEQ ID NO: 0051) |
| | | | | | 213.72] | | | | agcagaagccttcata[C/A]aagtaagaatatgctt |
| 2 | TTN | 179391846 | p.I26892F | 6.45E-03 | 309.3[19.31 - | 0.00276 | 0.00001 | T/A | (SEQ ID NO: 0052) |
| | | | | | 4954.45] | | | | tgtacgtccatgatga[T/A]cagggttgtcaggtca |
| 2 | TTN | 179396928 | p.R25740Q | 8.77E-02 | 11.31[1.53 - | 0.00276 | 0.00024 | C/T | (SEQ ID NO: 0053) |
| | | | | | 83.42] | | | | tgtcacttctctttgt[C/T]gccttgatttctttct |
| 2 | TTN | 179396965 | p.M25728L | 2.26E-02 | 1.05[6.13 - | 0.00276 | 0.00005 | T/G | (SEQ ID NO: 0054) |
| | | | | | 425.12] | | | | ttttcctcctttgaca[T/G]gaagtcaagttcgctt |
| 2 | TTN | 179397327 | p.A25607V | 2.17E-04 | 123.83[23.95 - | 0.00552 | 0.00004 | G/A | (SEQ ID NO: 0055) |
| | | | | | 640.33] | | | | ctctgttcttttcatt[G/A]ctaagtagtcatcaat |
| 2 | TTN | 179398405 | p.K25248X | 3.28E-03 | Inf | 0.00276 | not seen | T/A | (SEQ ID NO: 0056) |
| | | | | | | | | | aattggtaaagaccct[T/A]gtctgactcaaatgtg |
| 2 | TTN | 179399539 | p.I24870V | 2.88E-02 | 38.51[4.8 - | 0.00276 | 0.00007 | T/C | (SEQ ID NO: 0057) |
| | | | | | 308.73] | | | | atgtcaaagtgtccaa[T/C]attatgactgtgtaaa |
| 2 | TTN | 179399634 | p.R24838L | 3.28E-03 | Inf | 0.00276 | not seen | C/A | (SEQ ID NO: 0058) |
| | | | | | | | | | agcacttgtgttaatg[C/A]gctcaaatatgtcaag |
| 2 | TTN | 179403888 | p.G23860D | 9.69E-03 | 154.25[13.96 - | 0.00276 | 0.00002 | C/T | (SEQ ID NO: 0059) |
| | | | | | 1704.88] | | | | tgtgactctagaacca[C/T]catcatctttgggccg |
| 2 | TTN | 179407425 | p.G23321R | 2.89E-02 | 38.46[4.8 - | 0.00276 | 0.00007 | C/T | (SEQ ID NO: 0060) |
| | | | | | 308.29] | | | | gtttctgaagtagttc[C/T]ggtaacattcttcaat |
| 2 | TTN | 179410271 | p.R22791C | 3.24E-03 | Inf | 0.00276 | 0.00000 | G/A | (SEQ ID NO: 0061) |
| | | | | | | | | | tagtctttgttgacac[G/A]tgtccagcgcaggctc |
| 2 | TTN | 179411970 | p.R22363S | 1.61E-02 | 76.96[8.58 - | 0.00276 | 0.00004 | G/T | (SEQ ID NO: 0062) |
| | | | | | 690.25] | | | | tagggttcttcccaac+G/T+aatagacatggcattg |
| 2 | TTN | 179417691 | p.T20914I | 9.73E-03 | 153.65[13.9 - | 0.00276 | 0.00002 | G/A | (SEQ ID NO: 0063) |
| | | | | | 1698.2] | | | | tgacacgacagaccat[G/A]ttctcttggtggcatc |
| 2 | TTN | 179418306 | p.R20744Q | 2.94E-01 | 2.9[0.4 - | 0.00276 | 0.00095 | C/T | (SEQ ID NO: 0064) |
| | | | | | 20.83] | | | | gtttacagcagatacc[C/T]ggaagtagtaattgac |
| 2 | TTN | 179418418 | p.E20707K | 1.36E-01 | 6.99[0.96 - | 0.00276 | 0.00040 | C/T | (SEQ ID NO: 0065) |
| | | | | | 50.85] | | | | tctccttgtcttatct[C/T]gacaacatacccagta |
| 2 | TTN | 179419708 | p.T20428M | 6.49E-03 | 307.37[19.19 - | 0.00276 | 0.00001 | G/A | (SEQ ID NO: 0066) |
| | | | | | 4923.57] | | | | tatagatgcgaggtcc[G/A]tggtattttcaacaca |
| 2 | TTN | 179421656 | p.R20344C | 1.29E-02 | 102.58[10.64 - | 0.00276 | 0.00003 | G/A | (SEQ ID NO: 0067) |
| | | | | | 988.46] | | | | ttcctagcaaagacac[G/A]gaattcatactgggaa |
| 2 | TTN | 179421791 | p.G20299S | 2.09E-01 | 4.33[0.6 - | 0.00276 | 0.00064 | C/T | (SEQ ID NO: 0068) |
| | | | | | 31.25] | | | | cctgtaatcttgctac[C/T]tccatcgaaagctggt |
| 2 | TTN | 179425108 | p.G19519V | 3.28E-03 | Inf | 0.00276 | not seen | C/A | (SEQ ID NO: 0069) |
| | | | | | | | | | cctttcaattatatat[C/A]cagatatttcactacc |
| 2 | TTN | 179425543 | p.R19374Q | 1.29E-02 | 102.59[10.65 - | 0.00276 | 0.00003 | C/T | (SEQ ID NO: 0070) |
| | | | | | 988.54] | | | | attaacggccacagac[C/T]gagtgccggcaacatt |
| 2 | TTN | 179425744 | p.G19307E | 1.02E-01 | 9.6[1.31 - | 0.00276 | 0.00029 | C/T | (SEQ ID NO: 0071) |
| | | | | | 70.45] | | | | tatcttaaggacctct[C/T]cagctttgacaacaat |
| 2 | TTN | 179425757 | p.V19303F | 3.28E-03 | Inf | 0.00276 | not seen | C/A | (SEQ ID NO: 0072) |
| | | | | | | | | | tctccagctttgacaa[C/A]aataacgtctcggaac |
| 2 | TTN | 179425882 | p.R19261Q | 1.19E-01 | 8.08[1.11 - | 0.00276 | 0.00034 | C/T | (SEQ ID NO: 0073) |
| | | | | | 59.01] | | | | attccttgcaaaaacc[C/T]ggaattcataacgctg |
| 2 | TTN | 179427343 | p.R18774Q | 1.78E-01 | 5.18[0.72 - | 0.00276 | 0.00053 | C/T | (SEQ ID NO: 0074) |
| | | | | | 37.49] | | | | attggaagccaagact[C/T]ggaagtagtaagaaca |
| 2 | TTN | 179427544 | p.N18707I | 8.77E-02 | 11.31[1.53 - | 0.00276 | 0.00024 | T/A | (SEQ ID NO: 0075) |
| | | | | | 83.47] | | | | ttctcttatggtcaaa[T/A]tcacaggggcacttgg |
| 2 | TTN | 179428061 | p.A18535T | 2.30E-01 | 3.88[0.54 - | 0.00276 | 0.00071 | C/T | (SEQ ID NO: 0076) |
| | | | | | 27.96] | | | | tagcctttaacaggtg[C/T]gccaccatcataaatt |
| 2 | TTN | 179428837 | p.R18276Q | 2.26E-02 | 51.15[6.14 - | 0.00276 | 0.00005 | C/T | (SEQ ID NO: 0077) |
| | | | | | 425.93] | | | | ttgtcctccatcagtc[C/T]gtatacagtctttgac |
| 2 | TTN | 179429387 | p.P18093A | 2.32E-02 | 8.8[2.15 - | 0.00552 | 0.00063 | G/C | (SEQ ID NO: 0078) |
| | | | | | 36.02] | | | | tctgacactttgctag[G/C]cttgccaatgccaaca |
| 2 | TTN | 179431776 | p.I17296M | 6.49E-03 | 307.28[19.18 - | 0.00276 | 0.00001 | T/C | (SEQ ID NO: 0079) |
| | | | | | 4922.06] | | | | atttgttgacagccat[T/C]atacggaaaacatatt |
| 2 | TTN | 179437555 | p.R15370H | 6.51E-03 | 306.62[19.14 - | 0.00276 | 0.00001 | C/T | (SEQ ID NO: 0080) |
| | | | | | 4911.59] | | | | tatagtaacaactttg[C/T]gcaggtcagcatccag |
| 2 | TTN | 179437832 | p.A15278T | 9.70E-03 | 153.99[13.93 - | 0.00276 | 0.00002 | C/T | (SEQ ID NO: 0081) |
| | | | | | 1702.03] | | | | ataggtttattccaag[C/T]gattgaaatggatgat |
| 2 | TTN | 179437868 | p.R15266C | 1.29E-02 | 102.58[10.65 - | 0.00276 | 0.00003 | G/A | (SEQ ID NO: 0082) |
| | | | | | 988.47] | | | | cttgtatccagaacac[G/A]tgggttacctggtggt |
| 2 | TTN | 179437913 | p.K15251E | 3.28E-03 | Inf | 0.00276 | 0.00000 | T/C | (SEQ ID NO: 0083) |
| | | | | | | | | | acagtgtcacaagcct[T/C]gtaaaatggactggta |
| 2 | TTN | 179438938 | p.A14909V | 3.28E-03 | Inf | 0.00276 | not seen | G/A | (SEQ ID NO: 0084) |
| | | | | | | | | | aatgttgctgaagttg[G/A]ccttcagccaccgtcc |
| 2 | TTN | 179438948 | p.W14906R | 3.26E-03 | Inf | 0.00276 | 0.00000 | A/G | (SEQ ID NO: 0085) |
| | | | | | | | | | aagttggccttcagcc[A/G]ccgtccattaggaagg |
| 2 | TTN | 179439018 | p.K14882N | 6.50E-03 | 307.04[19.17 - | 0.00276 | 0.00001 | C/G | (SEQ ID NO: 0086) |
| | | | | | 4918.24] | | | | ccccagtatattcagg[C/G]ttagcccatttaagtg |
| 2 | TTN | 179439070 | p.K14865I | 5.68E-02 | 18.12[2.4 - | 0.00276 | 0.00015 | T/A | (SEQ ID NO: 0087) |
| | | | | | 136.49] | | | | atttagaggtactggt[T/A]ttccaggtgggtcaat |
| 2 | TTN | 179439085 | p.I14860T | 3.28E-03 | Inf | 0.00276 | not seen | A/G | (SEQ ID NO: 0088) |
| | | | | | | | | | ttttccaggtgggtca[A/G]tgggatccagagccaa |
| 2 | TTN | 179440186 | p.H14493R | 1.30E-02 | 102.33[10.62 - | 0.00276 | 0.00003 | T/C | (SEQ ID NO: 0089) |
| | | | | | 986.05] | | | | cttgctgccaccatcg[T/C]gtttgggcttaggcca |
| 2 | TTN | 179441724 | p.R14048Q | 6.33E-02 | 16.12[2.15 - | 0.00276 | 0.00017 | C/T | (SEQ ID NO: 0090) |
| | | | | | 120.71] | | | | gtttacagctgagacc[C/T]ggaagatgtactcatt |
| 2 | TTN | 179443660 | p.Q13634H | 1.61E-02 | 76.94[8.58 - | 0.00276 | 0.00004 | C/G | (SEQ ID NO: 0091) |
| | | | | | 690.03] | | | | ctctaaaggtggtttt[C/G]tggacagctgaggcgc |
| 2 | TTN | 179446785 | p.K13039T | 3.28E-03 | Inf | 0.00276 | not seen | T/G | (SEQ ID NO: 0092) |
| | | | | | | | | | aggttttctgttgacc[T/G]tagtccagttaacagc |
| 2 | TTN | 179447099 | p.F12963L | 3.28E-03 | Inf | 0.00276 | not seen | G/C | (SEQ ID NO: 0093) |
| | | | | | | | | | tttcagcacaaatacg[G/C]aactgatactcatggc |
| 2 | TTN | 179448375 | p.P12780L | 6.02E-02 | 17.01[2.26 - | 0.00276 | 0.00016 | G/A | (SEQ ID NO: 0094) |
| | | | | | 127.75] | | | | atcagaaggttcagaa[G/A]gtgggctaatgtttac |
| 2 | TTN | 179448393 | p.A12774V | 1.51E-01 | 6.25[0.86 - | 0.00276 | 0.00044 | G/A | (SEQ ID NO: 0095) |
| | | | | | 45.4] | | | | tgggctaatgtttacc[G/A]cggtcctggcaatagc |
| 2 | TTN | 179452021 | p.R12241H | 4.16E-02 | 25.5[3.31 - | 0.00276 | 0.00011 | C/T | (SEQ ID NO: 0096) |
| | | | | | 196.62] | | | | ctttctgtctgcctca[C/T]gtttctccacgatata |
| 2 | TTN | 179453343 | p.R11972C | 4.44E-02 | 23.89[3.1 - | 0.00276 | 0.00012 | G/A | (SEQ ID NO: 0097) |
| | | | | | 184.21] | | | | ttttctgccttgacac[G/A]gaactgatattcatgg |
| 2 | TTN | 179453585 | p.I11891T | 6.48E-03 | 307.88[19.22 - | 0.00276 | 0.00001 | A/G | (SEQ ID NO: 0098) |
| | | | | | 4931.73] | | | | atacttggttttggct[A/G]tgactggtttactttc |
| 2 | TTN | 179453906 | p.T11784M | 9.71E-03 | 153.94[13.93 - | 0.00276 | 0.00002 | G/A | (SEQ ID NO: 0099) |
| | | | | | 1701.51] | | | | cacaaaactgccagcc[G/A]tgttagttgccgtaac |
| 2 | TTN | 179458451 | p.V10461I | 9.91E-02 | 9.91[1.35 - | 0.00276 | 0.00028 | C/T | (SEQ ID NO: 0100) |
| | | | | | 72.76] | | | | gtcactggcatccaga[C/T]gtctttacccacttcc |
| 2 | TTN | 179465779 | p.C9553R | 3.27E-03 | Inf | 0.00276 | 0.00000 | A/G | (SEQ ID NO: 0101) |
| | | | | | | | | | gggtcccatgcaaggc[A/G]ctcaacaatatagtgg |
| 2 | TTN | 179469622 | p.R9000H | 2.91E-02 | 38.13[4.76 - | 0.00276 | 0.00007 | C/T | (SEQ ID NO: 0102) |
| | | | | | 305.68] | | | | tcttggtggggatggg[C/T]ggtctggaaaggaatc |
| 2 | TTN | 179470215 | p.R8871H | 2.89E-02 | 38.34[4.78 - | 0.00276 | 0.00007 | C/T | (SEQ ID NO: 0103) |
| | | | | | 307.31] | | | | attgacagctttgaca[C/T]ggaactcatacatttg |
| 2 | TTN | 179473176 | p.I8413M | 2.71E-02 | 41.65[5.11 - | 0.00276 | 0.00007 | A/C | (SEQ ID NO: 0104) |
| | | | | | 339.4] | | | | tggtaacatcttccac+A/C+atgggcttatctggtg |
| 2 | TTN | 179474228 | p.S82051 | 2.67E-01 | 3.25[0.45 - | 0.00276 | 0.00085 | C/A | (SEQ ID NO: 0105) |
| | | | | | 23.37] | | | | aggtgatccagaaata[C/A]ttgcatcaagtgctat |
| 2 | TTN | 179474255 | p.R8196Q | 7.30E-02 | 13.8[1.86 - | 0.00276 | 0.00020 | C/T | (SEQ ID NO: 0106) |
| | | | | | 102.67] | | | | tgctatttcatcacct[C/T]gtttcacttctaggct |
| 2 | TTN | 179475789 | p.G7958R | 3.25E-03 | Inf | 0.00276 | 0.00000 | C/T | (SEQ ID NO: 0107) |
| | | | | | | | | | gtaatggtataaattc[C/T]ggcatctgcacggaca |
| 2 | TTN | 179475791 | p.A7957V | 3.25E-03 | Inf | 0.00276 | 0.00000 | G/A | (SEQ ID NO: 0108) |
| | | | | | | | | | aatggtataaattccg[G/A]catctgcacggacact |
| 2 | TTN | 179475902 | p.T7920I | 4.15E-02 | 25.6[3.32 - | 0.00276 | 0.00011 | G/A | (SEQ ID NO: 0109) |
| | | | | | 197.38] | | | | tttatgccaactaaca[G/A]ttggaactgggacagc |
| 2 | TTN | 179478597 | p.W7406C | 1.99E-01 | 4.57[0.63 - | 0.00276 | 0.00061 | C/A | (SEQ ID NO: 0110) |
| | | | | | 33.02] | | | | catcatctggctcaca[C/A]catgtgagagtcactg |
| 2 | TTN | 179480434 | p.R7067C | 6.49E-03 | 307.34[19.19 - | 0.00276 | 0.00001 | G/A | (SEQ ID NO: 0111) |
| | | | | | 4923.04] | | | | gggccacatttgttac[G/A]agcacaaactttaaat |
| 2 | TTN | 179485598 | p.I6182L | 1.63E-02 | 76.37[8.51 - | 0.00276 | 0.00004 | T/G | (SEQ ID NO: 0112) |
| | | | | | 684.9] | | | | tttgaactatcaaata[T/G]agcttcttcatttctg |
| 2 | TTN | 179485612 | p.R6177K | 3.27E-03 | Inf | 0.00276 | 0.00000 | C/T | (SEQ ID NO: 0113) |
| | | | | | | | | | tatagcttcttcattt[C/T]tgaaccatttggcttt |
| 2 | TTN | 179494968 | p.P5696S | 6.28E-02 | 5.01[1.24 - | 0.00552 | 0.00111 | G/A | (SEQ ID NO: 0114) |
| | | | | | 20.35] | | | | aatatacaatacttac[G/A]cttaactcggaggtgg |
| 2 | TTN | 179496931 | p.S5499T | 1.07E-02 | 13.48[3.26 - | 0.00552 | 0.00041 | A/T | (SEQ ID NO: 0115) |
| | | | | | 55.83] | | | | tctactctaatttggg[A/T]ggtgtcatcaatagac |
| 2 | TTN | 179499158 | p.D5052V | 3.28E-03 | Inf | 0.00276 | not seen | T/A | (SEQ ID NO: 0116) |
| | | | | | | | | | atagaccccttcatca[T/A]caaattgagaatcatt |
| 2 | TTN | 179499293 | p.R5007Q | 6.50E-03 | 307.12[19.17 - | 0.00276 | 0.00001 | C/T | (SEQ ID NO: 0117) |
| | | | | | 4919.58] | | | | gaggacacattcgaat[C/T]gagcctgtcgcctttc |
| 2 | TTN | 179517039 | p.K11548E | 9.67E-03 | 154.51[13.98 - | 0.00276 | 0.00002 | T/C | (SEQ ID NO: 0118) |
| | | | | | 1707.76] | | | | ggaggcactggcactt[T/C]cttttcaggaacaact |
| 2 | TTN | 179539074 | p.I11185V | 1.72E-02 | 72.29[8.06 - | 0.00276 | 0.00004 | T/C | (SEQ ID NO: 0119) |
| | | | | | 648.38] | | | | tcttcaacttcctcta[T/C]gctaggtggttcttct |
| 2 | TTN | 179547564 | p.R10668P | 6.48E-03 | 307.78[19.21 - | 0.00276 | 0.00001 | C/G | (SEQ ID NO: 0120) |
| | | | | | 4930.05] | | | | ctcatattcttcttcc[C/G]gttgtactgaaacagc |
| 2 | TTN | 179553849 | p.K10359E | 9.98E-02 | 9.83[1.34 - | 0.00276 | 0.00028 | T/C | (SEQ ID NO: 0121) |
| | | | | | 72.18] | | | | tctgggacgggtttct[T/C]aggcagagctggcact |
| 2 | TTN | 179559325 | NM_0012568 | 1.14E-02 | 174.55[10.9 - | 0.00276 | 0.00002 | C/G | (SEQ ID NO: 0122) |
| | | | 50:exon116:c. | | 2796.03] | | | | aacacaaagatgtata[C/G]ctttcacttcaataac |
| | | | 30475 + 1G > C, | | | | | | |
| | | | NM_0012675 | | | | | | |
| | | | 50:exon118:c. | | | | | | |
| | | | 31426 + 1G > C, | | | | | | |
| | | | NM_133378: | | | | | | |
| | | | exon115: | | | | | | |
| | | | c.27694 + 1G > C | | | | | | |
| 2 | TTN | 179578720 | p.I8572V | 3.28E-03 | Inf | 0.00276 | not seen | T/C | (SEQ ID NO: 0123) |
| | | | | | | | | | ggtgctacattgatga[T/C]cttaaggccggatact |
| 2 | TTN | 179578821 | p.G8538V | 1.29E-02 | 102.73[10.66 - | 0.00276 | 0.00003 | C/A | (SEQ ID NO: 0124) |
| | | | | | 989.95] | | | | actgagttcaggggtg[C/A]cagctactgtacactc |
| 2 | TTN | 179580418 | p.G8258R | 6.68E-03 | 298.93[18.66 - | 0.00276 | 0.00001 | C/T | (SEQ ID NO: 0125) |
| | | | | | 4788.32] | | | | ttgatttctggagacc[C/T]accgattttgcattca |
| 2 | TTN | 179580495 | p.P8232H | 4.45E-02 | 24.4[3.08 - | 0.00276 | 0.00011 | G/T | (SEQ ID NO: 0126) |
| | | | | | 193.13] | | | | cttcttaatgaacctg[G/T]gtggttctatggaacc |
| 2 | TTN | 179582421 | p.Y8077D | 9.76E-03 | 153.18[13.86 - | 0.00276 | 0.00002 | A/C | (SEQ ID NO: 0127) |
| | | | | | 1693.03] | | | | agaaccccatccttgt[A/C]ccaagacacttgaaga |
| 2 | TTN | 179582456 | p.R8065H | 1.54E-01 | 6.09[0.84 - | 0.00276 | 0.00045 | C/T | (SEQ ID NO: 0128) |
| | | | | | 44.19] | | | | ttctgagccattgatg[C/T]ggcattcaaatgcaac |
| 2 | TTN | 179582514 | p.A8046S | 3.34E-03 | Inf | 0.00276 | 0.00000 | C/A | (SEQ ID NO: 0129) |
| | | | | | | | | | tctttcagttttcttg[C/A]aaagaaaggtggaagt |
| 2 | TTN | 179583194 | p.Q7896H | 2.25E-02 | 51.21[6.15 - | 0.00276 | 0.00005 | T/G | (SEQ ID NO: 0130) |
| | | | | | 426.44] | | | | tactgcatctctcaga[T/G]tgtgaaataagatact |
| 2 | TTN | 179583313 | p.V7857fs | 3.69E-13 | Inf | 0.01381 | not seen | C/CA | (SEQ ID NO: 0131) |
| | | | | | | | | | tcagccaatcttttca[c/ca]aaaggtggctggttc |
| 2 | TTN | 179583445 | p.C7844Y | 6.49E-03 | 307.49[19.2 - | 0.00276 | 0.00001 | C/T | (SEQ ID NO: 0132) |
| | | | | | 4925.43] | | | | aaaaagatgtgtggta[C/T]aggaagcactgccagc |
| 2 | TTN | 179583571 | p.V7802A | 3.25E-03 | Inf | 0.00276 | 0.00000 | A/G | (SEQ ID NO: 0133) |
| | | | | | | | | | gcatgactccccaggc[A/G]ccagttccctgctgcc |
| 2 | TTN | 179583950 | p.I7739M | 6.50E-03 | 306.87[19.16 - | 0.00276 | 0.00001 | G/A | (SEQ ID NO: 0134) |
| | | | | | 4915.58] | | | | attggcagccacacac[G/A]tgtatatgcctgtgtc |
| 2 | TTN | 179584336 | p.F7644L | 3.28E-03 | Inf | 0.00276 | not seen | A/T | (SEQ ID NO: 0135) |
| | | | | | | | | | cactgtttttcacttc[A/T]aagctatataatcctt |
| 2 | TTN | 179584782 | p.S7546P | 3.28E-03 | Inf | 0.00276 | not seen | A/G | (SEQ ID NO: 0136) |
| | | | | | | | | | catatatattttccag[A/G]attagatgcttctgga |
| 2 | TTN | 179584925 | p.R7498Q | 1.94E-02 | 61.35[7.15 - | 0.00276 | 0.00005 | C/T | (SEQ ID NO: 0137) |
| | | | | | 526.4] | | | | gccctccactatggcc[C/T]gtaactcaacagcatc |
| 2 | TTN | 179586814 | p.V7209I | 3.28E-03 | Inf | 0.00276 | not seen | C/T | (SEQ ID NO: 0138) |
| | | | | | | | | | ctttctccagcaataa[C/T]atctatagatacaggc |
| 2 | TTN | 179588157 | p.L6907fs | 3.28E-03 | Inf | 0.00276 | not seen | G/GA | (SEQ ID NO: 0139) |
| | | | | | | | | | aaacctttcacaaaga[g/ga]acgggtagtgcaaga |
| 2 | TTN | 179592455 | p.G6300D | 3.24E-03 | Inf | 0.00276 | 0.00000 | C/T | (SEQ ID NO: 0140) |
| | | | | | | | | | aatgaaacatttagga[C/T]ctgagacaagttccac |
| 2 | TTN | 179594113 | p.H5940R | 6.52E-03 | 306.26[19.12 - | 0.00276 | 0.00001 | T/C | (SEQ ID NO: 0141) |
| | | | | | 4905.73] | | | | gtcacacttggttata[T/C]ggaggttaaacacaga |
| 2 | TTN | 179596266 | p.R5426W | 2.27E-02 | 50.96[6.12 - | 0.00276 | 0.00005 | G/A | (SEQ ID NO: 0142) |
| | | | | | 424.39] | | | | gcagctgtgcctcccc[G/A]gagggagctggtactc |
| 2 | TTN | 179596668 | p.P5328L | 8.16E-02 | 12.23[1.65 - | 0.00276 | 0.00023 | G/A | (SEQ ID NO: 0143) |
| | | | | | 90.51] | | | | ctttaagacttcaatt[G/A]gcttaaattccttggt |
| 2 | TTN | 179597408 | p.D5143E | 3.28E-03 | Inf | 0.00276 | not seen | A/C | (SEQ ID NO: 0144) |
| | | | | | | | | | ctgagccaggcagaac[A/C]tcctttgagccgggtt |
| 2 | TTN | 179597437 | p.S5134G | 2.09E-02 | 56.93[6.64 - | 0.00276 | 0.00005 | T/C | (SEQ ID NO: 0145) |
| | | | | | 488.54] | | | | ggtttagttacaaaac[T/C]gggtggttctgaagaa |
| 2 | TTN | 179598563 | p.V4868I | 3.25E-03 | Inf | 0.00276 | 0.00000 | C/T | (SEQ ID NO: 0146) |
| | | | | | | | | | gcagcttgcagggtaa[C/T]ggtttgtcctcctagt |
| 2 | TTN | 179599121 | p.E4827K | 9.72E-03 | 153.7[13.91 - | 0.00276 | 0.00002 | C/T | (SEQ ID NO: 0147) |
| | | | | | 1698.78] | | | | acaacacattcatatt[C/T]accaacatctgcacta |
| 2 | TTN | 179599701 | p.V4667M | 8.12E-03 | 245.4[15.32 - | 0.00276 | 0.00001 | C/T | (SEQ ID NO: 0148) |
| | | | | | 3930.88] | | | | ggatccaccttcttca[C/T]gaaggatggtggctct |
| 2 | TTN | 179600313 | p.D4637H | 3.28E-03 | Inf | 0.00276 | not seen | C/G | (SEQ ID NO: 0149) |
| | | | | | | | | | acataggttcctgaat[C/G]tttcagtttggccaaa |
| 2 | TTN | 179600408 | p.S4605N | 9.70E-03 | 154.07[13.94 - | 0.00276 | 0.00002 | C/T | (SEQ ID NO: 0150) |
| | | | | | 1702.89] | | | | tttttgcccatctttg[C/T]tccacgtaactgtgac |
| 2 | TTN | 179602871 | p.Y4407C | 8.10E-02 | 2.32[1.67 - | 0.00276 | 0.00022 | T/C | (SEQ ID NO: 0151) |
| | | | | | 91.17] | | | | ggaagctttgcatgta[T/C]actcgccgcagtcaac |
| 2 | TTN | 179611064 | p.V5355L | 4.81E-02 | 21.76[2.85 - | 0.00276 | 0.00013 | C/G | (SEQ ID NO: 0152) |
| | | | | | 165.87] | | | | tccatatttggatcta[C/G]aaaattaaatggaaga |
| 2 | TTN | 179613763 | p.K4455R | 1.45E-02 | 11.41[2.77 - | 0.00552 | 0.00049 | T/C | (SEQ ID NO: 0153) |
| | | | | | 46.99] | | | | aattctatgcttcacc[T/C]ttttccccgggtagtg |
| 2 | TTN | 179616215 | p.T3638S | 4.76E-02 | 21.98[2.88 - | 0.00276 | 0.00013 | T/A | (SEQ ID NO: 0154) |
| | | | | | 167.59] | | | | ttaaagggagagccag[T/A]aaacctcaggtcaacc |
| 2 | TTN | 179616478 | p.R3550Q | 3.53E-02 | 30.66[3.91 - | 0.00276 | 0.00009 | C/T | (SEQ ID NO: 0155) |
| | | | | | 240.13] | | | | ctcagcttttataatc[C/T]gacgaagacctgttgg |
| 2 | TTN | 179620947 | NM_133437: | 3.34E-03 | Inf | 0.00276 | 0.00000 | A/G | (SEQ ID NO: 0156) |
| | | | exon45:c.10741 + | | | | | | agctaaaaatcaatta[A/G]ccaccttctacactta |
| | | | 2T > C, | | | | | | |
| | | | NM_001267550: | | | | | | |
| | | | exon47:c.11254 + | | | | | | |
| | | | 2T > C | | | | | | |
| 2 | TTN | 179621140 | p.F3517S | 3.20E-02 | 34.23[4.33 - | 0.00276 | 0.00008 | A/G | (SEQ ID NO: 0157) |
| | | | | | 270.87] | | | | ccaggttacatcaatg[A/G]aagaatcatcttttaa |
| 2 | TTN | 179628931 | p.R3363C | 9.67E-03 | 154.49[13.98 - | 0.00276 | 0.00002 | G/A | (SEQ ID NO: 0158) |
| | | | | | 1707.54] | | | | acccggcattgaaaac[G/A]ggctggctgcccttca |
| 2 | TTN | 179629535 | p.P3236L | 8.19E-02 | 12.19[1.65 - | 0.00276 | 0.00023 | G/A | (SEQ ID NO: 0159) |
| | | | | | 90.19] | | | | aacttggggcggttca[G/A]gagctaggagtaaatg |
| 2 | TTN | 179638756 | p.S2380F | 3.28E-03 | Inf | 0.00276 | not seen | G/A | (SEQ ID NO: 0160) |
| | | | | | | | | | ttccacactttccaag[G/A]agactttaacttcaag |
| 2 | TTN | 179638834 | p.R2354H | 1.61E-02 | 77.07[8.59 - | 0.00276 | 0.00004 | C/T | (SEQ ID NO: 0161) |
| | | | | | 691.25] | | | | taggatagcaatgggg[C/T]gggctgtgaaatatgg |
| 2 | TTN | 179639050 | p.I2314T | 6.48E-03 | 308.14[19.24 - | 0.00276 | 0.00001 | A/G | (SEQ ID NO: 0162) |
| | | | | | 4935.9] | | | | tccacgacgagatgta[A/G]ttgtatatttgccatt |
| 2 | TTN | 179639078 | p.E2305K | 3.20E-02 | 34.25[4.33 - | 0.00276 | 0.00008 | C/T | (SEQ ID NO: 0163) |
| | | | | | 271.01] | | | | ccattggatttaagct[C/T]cacatcattatgatac |
| 2 | TTN | 179640946 | p.R1882H | 1.29E-02 | 102.77[10.66 - | 0.00276 | 0.00003 | C/T | (SEQ ID NO: 0164) |
| | | | | | 990.27] | | | | gaaccttttgcttttg[C/T]ggatgagctgtccatt |
| 2 | TTN | 179641009 | p.R1861H | 5.66E-02 | 18.18[2.41 - | 0.00276 | 0.00015 | C/T | (SEQ ID NO: 0165) |
| | | | | | 136.95] | | | | gcctgttaccctgcag[C/T]ggaaccttgcagtctc |
| 2 | TTN | 179642449 | p.T1488A | 1.61E-02 | 77.04[8.59 - | 0.00276 | 0.00004 | T/C | (SEQ ID NO: 0166) |
| | | | | | 690.99] | | | | tcatgaaaccagaacg[T/C]ctctggcataggtcta |
| 2 | TTN | 179642583 | p.L1443P | 9.70E-03 | 154.06[13.94 - | 0.00276 | 0.00002 | A/G | (SEQ ID NO: 0167) |
| | | | | | 1702.77] | | | | ctcatctgtctcctcc[A/G]gcctacgtccagggga |
| 2 | TTN | 179643760 | p.R1350H | 9.73E-03 | 153.65[13.9 - | 0.00276 | 0.00002 | C/T | (SEQ ID NO: 0168) |
| | | | | | 1698.23] | | | | aagaacaacaggtata[C/T]gcagactagctctgcc |
| 2 | TTN | 179645895 | p.R1159L | 3.24E-03 | Inf | 0.00276 | 0.00000 | C/A | (SEQ ID NO: 0169) |
| | | | | | | | | | ttctccatgcttattg[C/A]gaacaacaatagtgta |
| 2 | TTN | 179648841 | p.V911I | 6.89E-02 | 14.69[1.97 - | 0.00276 | 0.00019 | C/T | (SEQ ID NO: 0170) |
| | | | | | 109.49] | | | | aagcgctcttcacgga[C/T]ggtggtgccagtgatg |
| 2 | TTN | 179650367 | p.K825E | 6.47E-03 | 308.67[19.27 - | 0.00276 | 0.00001 | T/C | (SEQ ID NO: 0171) |
| | | | | | 4944.33] | | | | tatccatgttctgtct[T/C]aggaacagaaattttt |
| 2 | TTN | 179659795 | p.S367P | 9.67E-03 | 154.51[13.98 - | 0.00276 | 0.00002 | A/G | (SEQ ID NO: 0172) |
| | | | | | 1707.79] | | | | gtcctgatctgagtag[A/G]ggttgtcagcgttgtc |
| 2 | TTN | 179664376 | p.H251R | 1.93E-02 | 61.77[7.2 - | 0.00276 | 0.00004 | T/C | (SEQ ID NO: 0173) |
| | | | | | 530.04] | | | | cctgggaggtgtttta[T/C]gtggcagctgttgccc |
| 2 | TTN | 179665163 | p.S181N | 1.47E-01 | 6.44[0.89 - | 0.00276 | 0.00043 | C/T | (SEQ ID NO: 0174) |
| | | | | | 46.77] | | | | agtagctcttccaacg[C/T]tattggtggcatttac |
| 3 | SCN5A | 38603958 | p.T1303M | 1.08E-01 | 9.05[1.24 - | 0.00276 | 0.00031 | G/A | (SEQ ID NO: 0175) |
| | | | | | 66.31] | | | | acggagtgcacgcagc[G/A]tccgcagtgacttgat |
| 3 | SCN5A | 38620907 | p.S1102Y | 1.20E-01 | 8.02[1.1 - | 0.00276 | 0.00035 | G/T | (SEQ ID NO: 0176) |
| | | | | | 58.6] | | | | ggcctcggcctcagag[G/T]aggcagtcgctgacac |
| 3 | SCN5A | 38651303 | p.A286S | 9.66E-03 | 154.69[14 - | 0.00276 | 0.00002 | C/A | (SEQ ID NO: 0177) |
| | | | | | 1709.78] | | | | ttggtgccgttgagcg[C/A]tgtgaagttgcgcacg |
| 4 | SRP72 | 57344588 | p.H229R | 1.82E-05 | 71.73[20.35 - | 0.00829 | 0.00012 | A/G | (SEQ ID NO: 0178) |
| | | | | | 252.78] | | | | gaactggccatcattc[A/G]tggtcagatggcttat |
| 4 | PDLIM3 | 186427772 | p.V185L | 1.66E-01 | 5.62[0.78 - | 0.00276 | 0.00049 | C/G | (SEQ ID NO: 0179) |
| | | | | | 40.71] | | | | tggagcatccggtaca[C/G]gtccgactcggggggc |
| 5 | SDHA | 223666 | p.A45T | 2.72E-01 | 3.18[0.44 - | 0.00276 | 0.00087 | G/A | (SEQ ID NO: 0180) |
| | | | | | 22.86] | | | | gaacaagagggcatct[G/A]ctaaagtttcagattc |
| 5 | SDHA | 224487 | p.Y55H | 9.68E-03 | 154.32[13.96 - | 0.00276 | 0.00002 | T/C | (SEQ ID NO: 0181) |
| | | | | | 1705.61] | | | | ccagatttctgctcag[T/C]atccagtagtggatca |
| 5 | SDHA | 256519 | p.A660G | 6.28E-02 | 16.24[2.17 - | 0.00276 | 0.00017 | C/G | (SEQ ID NO: 0182) |
| | | | | | 121.64] | | | | gccaccgtcccgccag[C/G]cattcgctcctactga |
| 6 | DSP | 7570791 | p.A566T | 1.13E-01 | 8.56[1.17 - | 0.00276 | 0.00032 | G/A | (SEQ ID NO: 0183) |
| | | | | | 62.63] | | | | cagggccatgacaatc[G/A]ccaaggtatgtcctca |
| 6 | DSP | 7576670 | p.R925Q | 3.22E-03 | 25.83[6.08 - | 0.00552 | 0.00022 | G/A | (SEQ ID NO: 0184) |
| | | | | | 109.71] | | | | tccaacacagtcatgc[G/A]gtttttgaatgagcag |
| 6 | DSP | 7579985 | p.Y1188H | 5.69E-02 | 18.09[2.4 - | 0.00276 | 0.00015 | T/C | (SEQ ID NO: 0185) |
| | | | | | 136.29] | | | | cacccggaagagagaa[T/C]atgaaaatgagctggc |
| 6 | DSP | 7581543 | p.Q1707R | 6.47E-03 | 308.53[19.26 - | 0.00276 | 0.00001 | A/G | (SEQ ID NO: 0186) |
| | | | | | 4942.12] | | | | gaaattgagaggctgc[A/G]gtctctcacagagaac |
| 6 | LAMA4 | 112430669 | p.V1808I | 1.10E-01 | 8.82[1.2 - | 0.00276 | 0.00031 | C/T | (SEQ ID NO: 0187) |
| | | | | | 64.53] | | | | caggagttgatgctta[C/T]ggcgccgctgaccagg |
| 6 | LAMA4 | 112457354 | p.A1122S | 3.28E-03 | Inf | 0.00276 | not seen | C/A | (SEQ ID NO: 0188) |
| | | | | | | | | | gcatcattaatttgag[C/A]tttctttaacgtatct |
| 6 | LAMA4 | 112462603 | p.V917I | 9.68E-03 | 154.32[13.96 - | 0.00276 | 0.00002 | C/T | (SEQ ID NO: 0189) |
| | | | | | 1705.7] | | | | gcaggccaggaactga[C/T]gggcttggagtccagg |
| 6 | LAMA4 | 112463419 | p.A850T | 5.66E-02 | 18.18[2.41 - | 0.00276 | 0.00015 | C/T | (SEQ ID NO: 0190) |
| | | | | | 136.99] | | | | ctcagagacgtgaagg[C/T]ctttaagtcatccata |
| 6 | LAMA4 | 112537586 | p.G94S | 8.97E-02 | 11.03[1.5 - | 0.00276 | 0.00025 | C/T | (SEQ ID NO: 0191) |
| | | | | | 81.26] | | | | acacagtatcctgagc[C/T]gtccaaacactcgttg |
| 6 | LAMA2 | 129371122 | p.C58G | 3.28E-03 | Inf | 0.00276 | not seen | T/G | (SEQ ID NO: 0192) |
| | | | | | | | | | cacgaccaatgcaaca[T/G]gtggagaaaaaggacc |
| 6 | LAMA2 | 129419331 | p.A137V | 3.23E-03 | Inf | 0.00276 | 0.00000 | C/T | (SEQ ID NO: 0193) |
| | | | | | | | | | taggtgttccagatcg[C/T]gtatgtgattgtgaag |
| 6 | LAMA2 | 129465201 | p.E265D | 3.28E-03 | Inf | 0.00276 | not seen | A/C | (SEQ ID NO: 0194) |
| | | | | | | | | | acaaagacccaagaga[A/C]attgaccccattgtca |
| 6 | LAMA2 | 129601267 | p.G838R | 2.24E-02 | 51.54[6.19 - | 0.00276 | 0.00005 | G/A | (SEQ ID NO: 0195) |
| | | | | | 429.17] | | | | tgatggatgccctgtc[G/A]ggtacacaggaccacg |
| 6 | LAMA2 | 129634075 | p.H1082Y | 1.38E-01 | 6.87[0.94 - | 0.00276 | 0.00040 | C/T | (SEQ ID NO: 0196) |
| | | | | | 49.99] | | | | aggccaatgcaactgt[C/T]atccaaaattctctgg |
| 6 | LAMA2 | 129636759 | p.P1232A | 3.81E-02 | 28.13[3.62 - | 0.00276 | 0.00010 | C/G | (SEQ ID NO: 0197) |
| | | | | | 218.43] | | | | agatctccatttggaa[C/G]ctttttattggaaact |
| 6 | LAMA2 | 129649445 | p.R1400P | 3.28E-03 | Inf | 0.00276 | not seen | G/C | (SEQ ID NO: 0198) |
| | | | | | | | | | ttgccgggattttatc[G/C]actgcgttctcaacca |
| 6 | LAMA2 | 129674460 | p.G1559S | 6.57E-02 | 15.46[2.07 - | 0.00276 | 0.00018 | G/A | (SEQ ID NO: 0199) |
| | | | | | 115.51] | | | | gggaaggaagtgtgac[G/A]gctgcaagcactggca |
| 6 | LAMA2 | 129704276 | p.V1657M | 1.20E-01 | 8.06[1.1 - | 0.00276 | 0.00034 | G/A | (SEQ ID NO: 0200) |
| | | | | | 58.84] | | | | tcattaggctaccaaa[G/A]tgacagcagatggcga |
| 6 | LAMA2 | 129777604 | p.M2278V | 1.02E-01 | 9.64[1.31 - | 0.00276 | 0.00029 | A/G | (SEQ ID NO: 0201) |
| | | | | | 70.76] | | | | tgtggatgcaaatgca[A/G]tgctgtttgttggtgg |
| 6 | LAMA2 | 129823907 | p.V2783G | 9.71E-03 | 153.87[13.92 - | 0.00276 | 0.00002 | T/G | (SEQ ID NO: 0202) |
| | | | | | 1700.71] | | | | tttgatgacaccaaag[T/G]taaaaaccggtatgta |
| 6 | LAMA2 | 129824406 | p.N2843S | 1.07E-01 | 9.08[1.24 - | 0.00276 | 0.00030 | A/G | (SEQ ID NO: 02031) |
| | | | | | 66.52] | | | | atccccaccaaaatca[A/G]tgatggccagtggcac |
| 6 | LAMA2 | 129833568 | p.T2973K | 3.28E-03 | Inf | 0.00276 | not seen | C/A | (SEQ ID NO: 0204) |
| | | | | | | | | | ttccgcacaactacaa[C/A]gactggagttcttctg |
| 6 | ECT2L | 139204009 | NM_001195037: | 3.51E-13 | Inf | 0.00000 | 0.00000 | G/T | (SEQ ID NO: 0205) |
| | | | exon15:c. | | | | | | gaaaactattgagaag+G/T+taaatgagtttcaatt |
| | | | 2028 + 1G > T, | | | | | | |
| | | | NM_001077706: | | | | | | |
| | | | exon16:c.2028 + | | | | | | |
| | | | 1G > T | | | | | | |
| 6 | SYNE1 | 152454445 | p.L8608P | 1.07E-05 | Inf | 0.00552 | not seen | A/G | (SEQ ID NO: 0206) |
| | | | | | | | | | gtctaataacttctcc[A/G]gttccttgatatgacg |
| 6 | SYNE1 | 152456276 | p.D8536A | 4.65E-02 | 5.96[1.47 - | 0.00552 | 0.00093 | T/G | (SEQ ID NO: 0207) |
| | | | | | 24.24] | | | | ctgaagtatctctgca[T/G]caaggttagaatcaat |
| 6 | SYNE1 | 152485345 | p.D7844N | 2.31E-03 | 31.01[7.22 - | 0.00552 | 0.00018 | C/T | (SEQ ID NO: 0208) |
| | | | | | 133.15] | | | | tccgagttacaggaat[C/T]gtagactattggcttg |
| 6 | SYNE1 | 152545711 | p.M7076T | 8.37E-02 | 11.89[1.61 - | 0.00276 | 0.00023 | A/G | (SEQ ID NO: 0209) |
| | | | | | 87.82] | | | | agagtatctggcctcc[A/G]tgaggtaactgtttat |
| 6 | SYNE1 | 152563538 | p.R6506Q | 1.66E-01 | 5.62[0.78 - | 0.00276 | 0.00049 | C/T | (SEQ ID NO: 0210) |
| | | | | | 40.72] | | | | attcagaccactcctc[C/T]gggagccaatgatcat |
| 6 | SYNE1 | 152646296 | p.R5123C | 2.56E-02 | 44.2[5.42 - | 0.00276 | 0.00006 | G/A | (SEQ ID NO: 0211) |
| | | | | | 360.16] | | | | gccacagctcgaaggc[G/A]tgtccagcgctgccag |
| 6 | SYNE1 | 152651968 | p.L4547I | 5.97E-02 | 17.17[2.29 - | 0.00276 | 0.00016 | G/T | (SEQ ID NO: 0213) |
| | | | | | 128.98] | | | | tgcagcgtaagtagaa[G/T]attttcatattctgga |
| 6 | SYNE1 | 152651971 | p.N4546H | 6.57E-02 | 15.46[2.07 - | 0.00276 | 0.00018 | T/G | (SEQ ID NO: 0214) |
| | | | | | 115.49] | | | | agcgtaagtagaagat[T/G]ttcatattctggagat |
| 6 | SYNE1 | 152655330 | p.E4132K | 7.78E-02 | 12.87[1.74 - | 0.00276 | 0.00022 | C/T | (SEQ ID NO: 0215) |
| | | | | | 95.38] | | | | tgttcaggcgattcct[C/T]cttctttgttaactta |
| 6 | SYNE1 | 152671343 | p.S3954T | 5.14E-03 | 20.01[4.77 - | 0.00552 | 0.00028 | C/G | (SEQ ID NO: 0216) |
| | | | | | 83.91] | | | | gattgtctccaggctg[C/G]ttgtctccaggaggtc |
| 6 | SYNE1 | 152702178 | p.T2998M | 9.68E-03 | 154.44[13.97 - | 0.00276 | 0.00002 | G/A | (SEQ ID NO: 0217) |
| | | | | | 1707.02] | | | | cactatctcctcatcc[G/A]tgttcttgccttccag |
| 6 | SYNE1 | 152708386 | p.F2777I | 1.61E-02 | 77.24[8.61 - | 0.00276 | 0.00004 | A/T | (SEQ ID NO: 0218) |
| | | | | | 692.73] | | | | tggtcaagcaggacga[A/T]cttctctttcagacct |
| 6 | SYNE1 | 152722394 | p.T2310M | 2.56E-02 | 44.15[5.42 - | 0.00276 | 0.00006 | G/A | (SEQ ID NO: 0219) |
| | | | | | 359.74] | | | | ttgtgtactttgagcc[G/A]tgaaatccttcagggt |
| 6 | SYNE1 | 152730273 | p.K2164R | 2.16E-01 | 4.18[0.58 - | 0.00276 | 0.00066 | T/C | (SEQ ID NO: 0220) |
| | | | | | 30.13] | | | | actactgtgaattttc[T/C]tcagctcagataacaa |
| 6 | SYNE1 | 152786487 | p.S620F | 3.24E-03 | Inf | 0.00276 | 0.00000 | G/A | (SEQ ID NO: 0221) |
| | | | | | | | | | atagcgatcccagtta[G/A]agatcacttcttccag |
| 6 | SYNE1 | 152792881 | p.T502A | 1.05E-05 | Inf | 0.00552 | 0.00000 | T/C | (SEQ ID NO: 0222) |
| | | | | | | | | | aggtgtagctctgatg[T/C]ggaggaaacaaaatga |
| 6 | SYNE1 | 152792883 | p.S501F | 1.05E-05 | Inf | 0.00552 | 0.00000 | G/A | (SEQ ID NO: 0223) |
| | | | | | | | | | gtgtagctctgatgtg[G/A]aggaaacaaaatgaaa |
| 6 | FGFR10P | 167438329 | p.L242X | 1.14E-10 | Inf | 0.00000 | not seen | T/A | (SEQ ID NO: 0224) |
| | | | | | | | | | tcggatgcacccccct[T/A]aaaaagtggactcagc |
| 9 | FXN | 71650816 | p.R40C | 2.24E-01 | 4.11[0.55 - | 0.00276 | 0.00067 | C/T | (SEQ ID NO: 0225) |
| | | | | | 30.57] | | | | cccactctgcggccgc[C/T]gtggcctgcgcaccga |
| x | DMD | 31165477 | p.R490H | 9.00E-03 | 221.29[13.81 - | 0.00276 | 0.00001 | C/T | (SEQ ID NO: 0226) |
| | | | | | 3544.67] | | | | catcctggcttccagg[C/T]ggcctttgtgttgacg |
| x | DMD | 32407669 | p.E1481D | 1.35E-02 | 110.52[10 - | 0.00276 | 0.00003 | T/G | (SEQ ID NO: 0227) |
| | | | | | 1221.56] | | | | gttccacactctttgt[T/G]tccaatgcaggcaagt |
| x | DMD | 32490382 | p.A942T | 1.79E-02 | 73.75[7.65 - | 0.00276 | 0.00004 | C/T | (SEQ ID NO: 0228) |
| | | | | | 710.64] | | | | acccatgtcctgatgg[C/T]actcatggtctcctga |
| x | DMD | 32591879 | p.R555C | 4.87E-02 | 22.05[2.81 - | 0.00276 | 0.00013 | G/A | (SEQ ID NO: 0229) |
| | | | | | 172.66] | | | | tgttcttcagtaagac[G/A]ttgccatttgagaagg |
| x | DMD | 32663135 | p.Q357H | 2.23E-02 | 9.07[2.2 - | 0.00552 | 0.00061 | T/G | (SEQ ID NO: 0230) |
| | | | | | 37.45] | | | | cattagaaatctctcc[T/G]tgtgcttgcaatgtgt |
| x | DMD | 32717327 | p.I237V | 2.68E-02 | 44.17[5.15 - | 0.00276 | 0.00006 | T/C | (SEQ ID NO: 0231) |
| | | | | | 379.04] | | | | tcctggatggcttcaa[T/C]gctcacttgttgaggc |

1. A method comprising applying a preeclampsia increased risk therapeutic to a patient having at least one preeclampsia increased risk associated biomarker in the DNA of said patient.

2. The method of claim 1, wherein said at least one preeclampsia increased risk associated biomarker defines the minor allele of a SNP.

3. The method of claim 1, wherein said at least one preeclampsia increased risk associated biomarker defines the minor allele of a biomarker of table 1 having an OR of greater than 1.0.

4. The method of claim 1, wherein said at least one preeclampsia increased risk associated biomarker defines a plurality of preeclampsia increased risk associated biomarkers, each preeclampsia increased risk associated biomarker defining the minor allele of a biomarker of table 1 having an OR of greater than 1.0.

5. The method of claim 1, wherein said preeclampsia increased risk therapeutic defines administering at least one of a blood pressure reduction medication, a corticosteroid, an anticonvulsant, early delivery of a fetus of said patient, and a combination thereof.

6. The method of claim 5, wherein said blood pressure reduction medication defines magnesium sulfate, and wherein said early delivery of a fetus of said patient defines a C-section delivery.

7. The method of claim 6, wherein said patient is preeclampsia asymptomatic.

8. The method of claim 1, wherein said patient defines a preeclampsia asymptomatic human female.

9. The method of claim 1, wherein said applying step is preceded by the step of obtaining clinical data (CD) of said patient.

10. The method of claim 1, wherein said applying step is preceded by the steps of:
identifying a preeclampsia asymptomatic human female subject,
obtaining clinical data (CD) and a biological sample of said subject,
assigning a raw clinical probability value (RCPV) to said CD,
assaying said sample,
detecting in said sample a plurality of preeclampsia increased risk associated biomarkers, each preeclampsia increased risk associated biomarker defining the minor allele of a biomarker of table 1 having an OR of greater than 1.0 resulting in preeclampsia increased risk associated biomarker data, and
performing a statistical analysis using said RCPV and said preeclampsia increased risk associated biomarker data to result in a preeclampsia condition prognosis of said subject of at least one of an increased risk of preeclampsia existence and an increased risk of preeclampsia predisposition.

11. A method comprising applying a preeclampsia decreased risk therapeutic to a patient having at least one preeclampsia decreased risk associated biomarker in the DNA of said patient.

12. The method of claim 11, wherein said at least one preeclampsia decreased risk associated biomarker defines the minor allele of a SNP.

13. The method of claim 11, wherein said at least one preeclampsia decreased risk associated biomarker defines the minor allele of a biomarker of table 1 having an OR of less than 1.0.

14. The method of claim 11, wherein said at least one preeclampsia decreased risk associated biomarker defines a plurality of preeclampsia decreased risk associated biomarkers, each preeclampsia decreased risk associated biomarker defining the minor allele of a biomarker of table 1 having an OR of less than 1.0.

15. The method of claim 11, wherein said preeclampsia decreased risk therapeutic defines canceling the administration of a preeclampsia increased risk therapeutic.

16. The method of claim 15, wherein said preeclampsia increased risk therapeutic defines at least one of a blood pressure reduction medication, a corticosteroid, an anticonvulsant, early delivery of a fetus of said patient, and a combination thereof.

17. The method of claim 16, wherein said blood pressure reduction medication defines magnesium sulfate, and wherein said early delivery of a fetus of said patient defines a C-section delivery.

18. The method of claim 11, wherein said patient defines a preeclampsia asymptomatic human female.

19. The method of claim 11, wherein said applying step is preceded by the step of obtaining clinical data (CD) of said patient.

20. The method of claim 11, wherein said applying step is preceded by the steps of:
identifying a preeclampsia asymptomatic human female subject,
obtaining clinical data (CD) and a biological sample of said subject,
assigning a raw clinical probability value (RCPV) to said CD,
assaying said sample,
detecting in said sample a plurality of preeclampsia decreased risk associated biomarkers, each preeclampsia decreased risk associated biomarker defining the minor allele of a biomarker of table 1 having an OR of less than 1.0 resulting in preeclampsia decreased risk associated biomarker data, and
performing a statistical analysis using said RCPV and said preeclampsia decreased risk associated biomarker data to result in a preeclampsia condition prognosis of said subject of at least one of a decreased risk of preeclampsia existence and a decreased risk of preeclampsia predisposition.

21. A method comprising applying a preeclampsia altered risk therapeutic to a patient having at least one preeclampsia altered risk associated biomarker in the DNA of said patient.

22. The method of claim 21, wherein said at least one preeclampsia altered risk associated biomarker defines the minor allele of a SNP.

23. The method of claim 21, wherein said preeclampsia altered risk defines at least one of a preeclampsia increased risk as compared to a control subject and a preeclampsia decreased risk as compared to a control subject, and wherein said preeclampsia altered risk therapeutic defines at least one of a preeclampsia increased risk therapeutic and a preeclampsia decreased risk therapeutic.

24. The method of claim 23, wherein said at least one preeclampsia increased risk associated biomarker defines the minor allele of a biomarker of table 1 having an OR of greater than 1.0, and wherein said at least one preeclampsia decreased risk associated biomarker defines the minor allele of a biomarker of table 1 having an OR of less than 1.0.

25. The method of claim 23, wherein said at least one preeclampsia increased risk associated biomarker defines a plurality of preeclampsia increased risk associated biomarkers, each preeclampsia increased risk associated biomarker defining the minor allele of a biomarker of table 1 having an OR of greater than 1.0, and wherein said at least one preeclampsia decreased risk associated biomarker defines a plurality of preeclampsia decreased risk associated biomarkers, each preeclampsia decreased risk associated biomarker defining the minor allele of a biomarker of table 1 having an OR of less than 1.0.

26. The method of claim 23, wherein said preeclampsia increased risk therapeutic defines administering at least one of a blood pressure reduction medication, a corticosteroid, an anticonvulsant, early delivery of a fetus of said patient, and a combination thereof, and wherein said preeclampsia decreased risk therapeutic defines canceling the administration of a preeclampsia increased risk therapeutic.

27. The method of claim 26, wherein said blood pressure reduction medication defines magnesium sulfate, and wherein said early delivery of a fetus of said patient defines a C-section delivery.

28. The method of claim 27, wherein said patient is preeclampsia asymptomatic.

29. The method of claim 21, wherein said patient defines a preeclampsia asymptomatic human female.

30. The method of claim 21, wherein said applying step is preceded by the step of obtaining clinical data (CD) of said patient.

31. The method of claim 21, wherein said applying step is preceded by the steps of:
identifying a preeclampsia asymptomatic human female subject,
obtaining clinical data (CD) and a biological sample of said subject,
assigning a raw clinical probability value (RCPV) to said CD,
assaying said sample,
detecting in said sample at least one of a plurality of preeclampsia increased risk associated biomarkers, each preeclampsia increased risk associated biomarker defining the minor allele of a biomarker of table 1 having an OR of greater than 1.0 resulting in preeclampsia increased risk associated biomarker data and a plurality of preeclampsia decreased risk associated biomarkers, each preeclampsia decreased risk associated biomarker defining the minor allele of a biomarker of table 1 having an OR of less than 1.0 resulting in preeclampsia decreased risk associated biomarker data, and
performing at least one of a statistical analysis using said RCPV and said preeclampsia increased risk associated biomarker data to result in a preeclampsia condition prognosis of said subject of at least one of an increased risk of preeclampsia existence and an increased risk of preeclampsia predisposition and a statistical analysis using said RCPV and said preeclampsia decreased risk associated biomarker data to result in a preeclampsia condition prognosis of said subject of at least one of a decreased risk of preeclampsia existence and a decreased risk of preeclampsia predisposition.
